SEQUENCE LISTING (25 sequences)

SEQ ID NO: 1 - DNA, 180 bp, synthetic construct
  source 1..180: mol_type = other DNA
ttgcgccacagagccgacgtgtacgtgtgtcggcgtcccctgaggttcagcacagaaaaaccggagcgcttagagtataactgctccatccgcggatggagcagttatactctaagcgctccggtttttctgtgctgaacctcaggggacgccgacacacgtacacgtcggctctgtggc

SEQ ID NO: 2 - DNA, 186 bp, synthetic construct
  source 1..186: mol_type = other DNA
gcaatggatagcgcattggacttcaaattcaaaggttgtgggttcgagtcccaccagagtcggtcctttttttgatccagcagggtggatgcccgcgggcatccaccctgctggatcaaaaaaaggaccgactctggtgggactcgaacccacaacctttgaatttgaagtccaatgcgctatcca

SEQ ID NO: 3 - DNA, 64 bp, synthetic construct
  source 1..64: mol_type = other DNA
tagagtctacctgctccatcctcggatggagcaggtagactctaagcgctccggtttttctgtg

SEQ ID NO: 4 - DNA, 119 bp, synthetic construct
  source 1..119: mol_type = other DNA
ctgaacctcaggggacgccgacacacgtacacgtcggctctgtggcggctctgtggcgcaatggatagcgcattggacttcaaattcaaaggttgtgggttcgagtcccaccagagtcg

SEQ ID NO: 5 - DNA, 153 bp, synthetic construct
  source 1..153: mol_type = other DNA
gggaaaaaaaggaccgactctggtgggactcgaacccacaacctttgaatttgaagtccaatgcgctatccattgcgccacagagccgccacagagccgacgtgtacgtgtgtcggcgtcccctgaggttcagcacagaaaaaccggagcgct

SEQ ID NO: 6 - DNA, 66 bp, synthetic construct
  source 1..66: mol_type = other DNA
gtcctttttttccccatcagtttttataaacttacacactcgtgtgtaagtttataaaaactgatg

SEQ ID NO: 7 - DNA, 54 bp, synthetic construct
  source 1..54: mol_type = other DNA
ctccaggagatagagtctacctgctccatcctcggatggagcaggtagactcta

SEQ ID NO: 8 - DNA, 160 bp, synthetic construct
  source 1..160: mol_type = other DNA
tctcctggagagcgctccggtttttctgtgctgaacctcaggggacgccgacacacgtacacgtcggctctgtggcggctctgtggcgcaatggatagcgcattggacttcaaattcaaaggttgtgggttcgagtcccaccagagtcggtccttttttt

SEQ ID NO: 9 - DNA, 160 bp, synthetic construct
  source 1..160: mol_type = other DNA
ccgtagagcgaaaaaaaggaccgactctggtgggactcgaacccacaacctttgaatttgaagtccaatgcgctatccattgcgccacagagccgccacagagccgacgtgtacgtgtgtcggcgtcccctgaggttcagcacagaaaaaccggagcgct

SEQ ID NO: 10 - DNA, 62 bp, synthetic construct
  source 1..62: mol_type = other DNA
cgctctacgggatccagcagggtggatgcccacactcgtgtgggcatccaccctgctggatc

SEQ ID NO: 11 - DNA, 54 bp, synthetic construct; identical to SEQ ID NO: 7
  source 1..54: mol_type = other DNA
  modified_base 32: mod_base = OTHER; note = DBCO-TEG (Dibenzo-bicyclo-octyne (DBCO) structure with a Triethyleneglycol (TEG) spacer)

SEQ ID NO: 12 - DNA, 62 bp, synthetic construct; identical to SEQ ID NO: 10
  source 1..62: mol_type = other DNA
  modified_base 36: mod_base = OTHER; note = DBCO-TEG (Dibenzo-bicyclo-octyne (DBCO) structure with a Triethyleneglycol (TEG) spacer)

SEQ ID NO: 13 - DNA, 159 bp, synthetic construct
  source 1..159: mol_type = other DNA
tctcctggagagcgctccggtttttctgtgctgaacctcaggggacgccgacacacgtacacgtcaccagaatggccgagtggttaaggcgttggacttcagatccaatggattcatatccgcgtgggttcgaaccccacttctggtagtccttttttt

SEQ ID NO: 14 - DNA, 159 bp, synthetic construct
  source 1..159: mol_type = other DNA
ccgtagagcgaaaaaaaggactaccagaagtggggttcgaacccacgcggatatgaatccattggatctgaagtccaacgccttaaccactcggccattctggtgacgtgtacgtgtgtcggcgtcccctgaggttcagcacagaaaaaccggagcgct

SEQ ID NO: 15 - DNA, 173 bp, synthetic construct
  source 1..173: mol_type = other DNA
tctcctggaggccaggggtgtggccatacaggtttatagtggttagtagagacaagtaagtgggtgcccctgtggcgcaatggatagcgcattggacttcaaatccaaaggttgcgggttcgagtcccgccaggggcggaccttttccccatcagtttttataaacttacaca

SEQ ID NO: 16 - DNA, 173 bp, synthetic construct
  source 1..173: mol_type = other DNA
ccgtagagcgtgtgtaagtttataaaaactgatggggaaaaggtccgcccctggcgggactcgaacccgcaacctttggatttgaagtccaatgcgctatccattgcgccacaggggcacccacttacttgtctctactaaccactataaacctgtatggccacacccctggc

SEQ ID NO: 17 - DNA, 183 bp, synthetic construct
  source 1..183: mol_type = other DNA
tctcctggaggccaggggtgtggccatacaggtttatagtggttagtagagacaagtaagtgggtgccaggatggccgagtggttaaggcgccggacttcagatccggtggactcatgtccgcccgggttcgaaccccggtcctggcagaccttttccccatcagtttttataaacttacaca

SEQ ID NO: 18 - DNA, 183 bp, synthetic construct
  source 1..183: mol_type = other DNA
ccgtagagcgtgtgtaagtttataaaaactgatggggaaaaggtctgccaggaccggggttcgaacccgggcggacatgagtccaccggatctgaagtccggcgccttaaccactcggccatcctggcacccacttacttgtctctactaaccactataaacctgtatggccacacccctggc

SEQ ID NO: 19 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttgtgggttcgagtcccaccagagtcggtccttttttt

SEQ ID NO: 20 - DNA, 149 bp, synthetic construct
  source 1..149: mol_type = other DNA
agcgctccggtttttctgtgctgaacctcaggggacgccgacacacgtacacgtcaccagaatggccgagtggttaaggcgttggacttcagatccaatggattcatatccgcgtgggttcgaaccccacttctggtagtccttttttt

SEQ ID NO: 21 - DNA, 163 bp, synthetic construct
  source 1..163: mol_type = other DNA
gccaggggtgtggccatacaggtttatagtggttagtagagacaagtaagtgggtgcccctgtggcgcaatggatagcgcattggacttcaaatccaaaggttgcgggttcgagtcccgccaggggcggaccttttccccatcagtttttataaacttacaca

SEQ ID NO: 22 - DNA, 173 bp, synthetic construct
  source 1..173: mol_type = other DNA
gccaggggtgtggccatacaggtttatagtggttagtagagacaagtaagtgggtgccaggatggccgagtggttaaggcgccggacttcagatccggtggactcatgtccgcccgggttcgaaccccggtcctggcagaccttttccccatcagtttttataaacttacaca

SEQ ID NO: 23 - DNA, 55 bp, synthetic construct
  source 1..55: mol_type = other DNA
gccaggggtgtggccatacaggtttatagtggttagtagagacaagtaagtgggt

SEQ ID NO: 24 - DNA, 35 bp, synthetic construct
  source 1..35: mol_type = other DNA
gaccttttccccatcagtttttataaacttacaca

SEQ ID NO: 25 - protein, 31 aa, synthetic construct
  MOD_RES 1: note = Azido lysine
LGGPSEVANKEPQQTAEGKEGKTRAKRDEDQ

(19) United States
(12) Patent Application Publication
(10) Pub. No.: US 2026/0234619 A1
(43) Pub. Date: Aug. 13, 2026

(54) AGENTS AND METHODS FOR MAKING CLOSED-END DNA THREAD MOLECULES

(71) Applicant: University of Rochester, Rochester, NY (US)

(72) Inventors: John D. Lueck, Rochester, NY (US); Joseph J. Porter, Rochester, NY (US)

(73) Assignee: University of Rochester, Rochester, NY (US)

(21) Appl. No.: 19/156,637

(22) PCT Filed: Feb. 22, 2024
(86) PCT No.: PCT/US2024/016802

§ 371 Date: Aug. 14, 2025

Related U.S. Application Data
(60) Provisional application No. 63/486,491, filed on Feb. 23, 2023.

(51) Int. Cl. C12N 15/113

(52) U.S. Cl. CPC C12N 15/113; C12N 2310/531; C12N 2310/532; C12N 2330/00

(57) ABSTRACT

This disclosure relates to agents and methods for making closed-end DNA thread (CEDT) molecules, as well as compositions and uses of CEDT molecules.

### CROSS REFERENCE TO RELATED APPLICATION

This application claims priority to U.S. Provisional Application No. 63/486,491 filed on Feb. 23, 2023. The content of the application is incorporated herein by reference in its entirety.

### STATEMENT REGARDING FEDERALLY SPONSORED RESEARCH

This invention was made with government support under HL153988 awarded by the National Institutes of Health. The government has certain rights in the invention.

### REFERENCE TO SEQUENCE LISTING

The present application is being filed along with a Sequence Listing in electronic format. The Sequence Listing is provided as a file entitled SeqList-161118-04501.xml, created on Feb. 20, 2023, which is 27,224 bytes in size. The information in the electronic format of the sequence listing is incorporated herein by reference in its entirety.

### FIELD OF THE INVENTION

This invention relates generally to agents and methods for making closed-end DNA thread (CEDT) molecules.

### BACKGROUND OF THE INVENTION

Nonsense mutations account for more than 10% of all genetic diseases and nearly 1,000 genetic human disorders, including cancer that affects about 300 million people worldwide. For example, about 22% of all cystic fibrosis patients have “class 1” premature termination codon (PTC) mutations (e.g., p.G542X, p.R553X, and p.W1282X), resulting in nearly complete loss of cystic fibrosis transmembrane conductance regulator (CFTR) function and severe clinical manifestations. Nonsense mutations change an amino acid codon to a PTC generally through a single-nucleotide substitution, resulting in defective truncated proteins and severe diseases.

Because of exceedingly high prevalence of nonsense-associated diseases and a unifying mechanism, there has been a concerted effort to develop PTC therapeutics. Aminoglycosides have been the primary focus of these efforts. However, ototoxicity and nephrotoxicity with extended use has restricted their use clinically. Synthetic aminoglycoside derivatives are currently being investigated to reduce off-target effects; yet, they often suffer low readthrough efficiency. Non-aminoglycoside small molecules (e.g., tylosin, Ataluren) have also been identified as promising PTC readthrough compounds with little toxicity. However, these approaches have a number of challenges yet to be overcome, including insertion of near-cognate tRNAs that often lead to the generation of a missense mutation at the site of the original PTC. Furthermore, some of the compounds have low efficiency of PTC suppression in human primary cells, which resulted in Ataluren failing phase 3 clinical trials.

Therefore, there is a need for agents and methods for treating diseases or disorders associated with or caused by nonsense mutations.

### SUMMARY OF THE INVENTION

This disclosure addresses the need mentioned above in a number of aspects. In one aspect, the disclosure provides an oligonucleotide set comprising: (a) a first hairpin oligonucleotide comprising from 5′ end to 3′ end: a first antisense strand, a first loop, and a first sense strand that is complementary to the first antisense strand; and (b) a second hairpin oligonucleotide comprising from 5′ end to 3′ end: a second sense strand, a second loop, and a second antisense strand that is complementary to the second sense strand, wherein the first sense strand and the second sense strand are adapted to be joined together to form a nucleic acid sequence encoding a RNA molecule.

In some embodiments, when the first sense strand and the second sense strand are joined, the first hairpin oligonucleotide and the second hairpin oligonucleotide form a closed-end DNA thread (CEDT) molecule, which is also called a picovector in some cases.

In some embodiments, the first hairpin oligonucleotide comprises a nucleic acid sequence encoding a tRNA leader. In some embodiments, the second hairpin oligonucleotide comprises a nucleic acid sequence encoding a RNA polymerase III termination signal.

In some embodiments, the oligonucleotide set further comprises a third sense strand and a third antisense strand having a sequence complementary to the third sense strand. In some embodiments, the first sense strand, the third sense strand, and the second sense strand are adapted to be joined together in the order to form a second nucleic acid sequence encoding the RNA molecule.

In another aspect, this disclosure provides an oligonucleotide set comprising: (i) a first hairpin oligonucleotide comprising from 5′ end to 3′ end: a first antisense strand, a first loop, and a first sense strand that is complementary to the first antisense strand; (ii) a second hairpin oligonucleotide comprising from 5′ end to 3′ end: a second sense strand, a second loop, and a second antisense strand that is complementary to the second sense strand; and (iii) a third sense strand and a third antisense strand having a sequence complementary to the third sense strand, wherein the first sense strand, the third sense strand, and the second sense strand are adapted to be joined together in the order to form a second nucleic acid sequence encoding the RNA molecule.

In some embodiments, when the first sense strand, the third sense strand, and the second sense strand are joined, the first hairpin oligonucleotide, the third sense and antisense strands, and the second hairpin oligonucleotide form a CEDT molecule.

In some embodiments, the third sense strand comprises a nucleic sequence encoding a tRNA leader. In some embodiments, the third sense strand comprises a nucleic sequence encoding a RNA polymerase III termination signal.

In some embodiments, the RNA molecule comprises tRNA. In some embodiments, the tRNA comprises an anti-codon edited-tRNA (ACE-tRNA). In some embodiments, the ACE-tRNA causes a ribosome to read through one or more stop codons during translation. In some embodiments, the one or more stop codons comprise a premature termination codon (PTC). Examples of the PTC include PTCs that result in disease or PTCs that result in nonsense-associated diseases. In some embodiments, the PTC is present in a nucleic acid sequence encoding cystic fibrosis transmembrane conductance regulator (CFTR).

In some embodiments, the tRNA is selected from the group consisting of Arg-tRNA-UGA, Gln-tRNA-UAA, Glnt-RNA-UAG, Trp-tRNA-UGA, Trp-tRNA-UAG, Glu-tRNA-UAA, Glu-tRNA-UAG, Cys-tRNA-UGA, Tyr-tRNA-UAG, Tyr-tRNA-UAA, Leu-tRNA-UGA, Leu-tRNA-UAG, Leu-tRNA-UAA, Lys-tRNA-UAG, Lys-tRNA-UGA, Ser-tRNA-UGA, Ser-tRNA-UAG, and Ser-tRNA-UAA.

In some embodiments, the nucleic acid sequence comprises a polynucleotide sequence of SEQ ID NO: 4, 8, 13, 15, and 17, or comprises a polynucleotide sequence having at least 85% sequence identity with a polynucleotide sequence of SEQ ID NO: 4, 8, 13, 15, and 17.

In some embodiments, the first hairpin oligonucleotide comprises a polynucleotide sequence of SEQ ID NO: 1, 3, 7, and 11, or comprises a polynucleotide sequence having at least 85% sequence identity with a polynucleotide sequence of SEQ ID NO: 1, 3, 7, and 11; and/or the second hairpin oligonucleotide comprises a polynucleotide sequence of SEQ ID NO: 2, 6, 10, and 12, or comprises a polynucleotide sequence having at least 85% sequence identity with a polynucleotide sequence of SEQ ID NO: 2, 6, 10, and 12.

In some embodiments, the nucleic acid sequence has a size of from 200 nucleotides to 1,000 nucleotides.

In some embodiments, the first loop or the second loop or another part of the oligonucleotide is linked with an agent. In some embodiments, the agent comprises a labeling agent, a peptide, a bioactive agent, or a combination thereof. In some embodiments, the labeling agent comprises any one of N-hydroxysuccinimide (NHS), thiol-maleimide, and azide-dibenzocyclooctyne (DBCO). Such an agent can be linked to the oligonucleotide via any suitable methods known in the art, such as bioorthogonal chemistry and click chemistry. Exemplary reactions may include native chemical ligation and the Staudinger ligation, copper-catalyzed azide-alkyne cycloaddition, strain-promoted [3+2] reactions, tetrazine ligation, metal-catalyzed coupling reactions, oxime and hydrazone ligations as well as photoinducible bioorthogonal reactions.

In some embodiments, the first hairpin oligonucleotide or the second hairpin oligonucleotide comprises one or more chemically modified nucleotides. In some embodiments, the one or more chemically modified nucleotides comprise a 2′-O-methyl-modified sugar moiety. In some embodiments, the one or more chemically modified nucleotides comprise a modified internucleoside linkage.

Also within the scope of this disclosure is a composition comprising the oligonucleotide set described herein.

In another aspect, this disclosure also provides a kit comprising the oligonucleotide set described herein and, optionally, a ligase. In some embodiments, the ligase is a T4 DNA ligase.

In yet another aspect, this disclosure further provides a method for making a CEDT molecule. In some embodiments, the method comprises: providing an oligonucleotide set described herein; and ligating components of the oligonucleotide set, thereby obtaining the CEDT molecule.

In some embodiments, the oligonucleotide set is synthesized chemically. In some embodiments, the oligonucleotide set is synthesized with chemically modified nucleotides.

In some embodiments, the CEDT is further linked to a labeling agent, a peptide, a bioactive agent, or a combination thereof.

In another aspect, this disclosure provides a CEDT molecule made according to the method described herein.

In yet another aspect, this disclosure additionally provides a method of treating a disease associated with a PTC in a subject in need thereof. In some embodiments, the method comprises administering to the subject the CEDT molecule described herein or a pharmaceutical composition thereof.

In some embodiments, the disease is selected from the group consisting of cystic fibrosis, Duchenne and Becker muscular dystrophies, retinoblastoma, neurofibromatosis, ataxia-telangiectasia, Tay-Sachs disease, Wilm's tumor, hemophilia A, hemophilia B, Menkes disease, Ullrich's disease, b-Thalassemia, type 2A and type 3 von Willebrand disease, Robinow syndrome, brachydactyly type B (shortening of digits and metacarpals), inherited susceptibility to mycobacterial infection, inherited retinal disease, inherited bleeding tendency, inherited blindness, congenital neurosensory deafness and colonic agangliosis and inherited neural develop-mental defect including neurosensory deafness, colonic agangliosis, peripheral neuropathy and central dysmyelinating leukodystrophy, Liddle's syndrome, xeroderma pigmentosum, Fanconi's anemia, anemia, hypothyroidism, p53-associated cancers, esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes, rickets, Hurler Syndrome, Dravet Syndrome, Spinal Muscular Dystrophy, Usher Syndrome, Aniridia, Choroideremia, Ocular Coloboma, Retinitis pigmentosa, dystrophic epidermolysis bullosa, Pseudoxanthoma elasticum, Alagille Snydrome, Waardenburg-Shah, infantile neuronal ceroid lipofuscinosis, Cystinosis, X-linked nephrogenic diabetes insipidus, McArdle's disease and Polycystic kidney disease.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if combinations of features are not found together in the same sentence, or paragraph, or section of this document. Other features and advantages of the invention will become apparent from the following detailed description. It should be understood, however, that the detailed 30 description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows an example scheme for ArgTGA CEDT production using synthetic hairpins (synthHP). This scheme is also referred to as a “1+1 scheme.”

FIG. 2 shows incorporation of biorthogonal reactive groups through ArgTGA synthHP production.

FIG. 3 shows an electrophoresis analysis following ArgTGA CEDT production by ligation of synthetic hairpins (synthHP).

FIG. 4 shows that ArgTGA CEDT synthHP rescues PTCs in vivo.

FIG. 5 shows quantitation of a 200-bp ArgTGA CEDT produced by ligation of synthetic hairpins (synthHP).

FIG. 6 shows an example scheme for ArgTGA CEDT production by ligation of synthetic hairpins (synthHP). This scheme is also referred to as a “2+2 scheme.”

FIG. 7 shows an example scheme for ArgTGA CEDT production by ligation of synthetic hairpins (synthHP). This scheme is also referred to as a “4P scheme.”

FIGS. 8A, 8B, and 8C shows production of labeled ACE-tRNA DNA picovectors and influence of labels on ACE-tRNA function. FIG. 8A shows a labeled ACE-tRNA picovector assembly scheme. FIG. 8B shows electrophoresis results of labeled ACE-tRNA DNA picovector products. FIG. 8C shows labeled ACE-tRNAArgTGA picovectors were effective in nonsense suppression.

### DETAILED DESCRIPTION OF THE INVENTION

This disclosure relates to novel agents and methods for making closed-end DNA thread (CEDT) molecules. CEDT molecules can be used for delivery and expression of RNA molecules, such as anti-codon edited-tRNAs (ACE-tRNAs). ACE-tRNAs are capable of reverting a nonsense mutation (e.g., a premature termination codon (PTC)) to an amino acid during the translation of mRNAs. Thus, ACE-tRNAs can be used to treat genetic diseases associated with nonsense mutations.

### Oligonucleotides and Methods for Making CEDT Molecules

### Oligonucleotide Sets for Making CEDT Molecules

Accordingly, the disclosure provides an oligonucleotide set comprising: (a) a first hairpin oligonucleotide comprising from 5′ end to 3′ end: a first antisense strand, a first loop, and a first sense strand that is complementary to the first antisense strand; and (b) a second hairpin oligonucleotide comprising from 5′ end to 3′ end: a second sense strand, a second loop, and a second antisense strand that is complementary to the second sense strand, wherein the first sense strand and the second sense strand are adapted to be joined together to form a nucleic acid sequence encoding a RNA molecule.

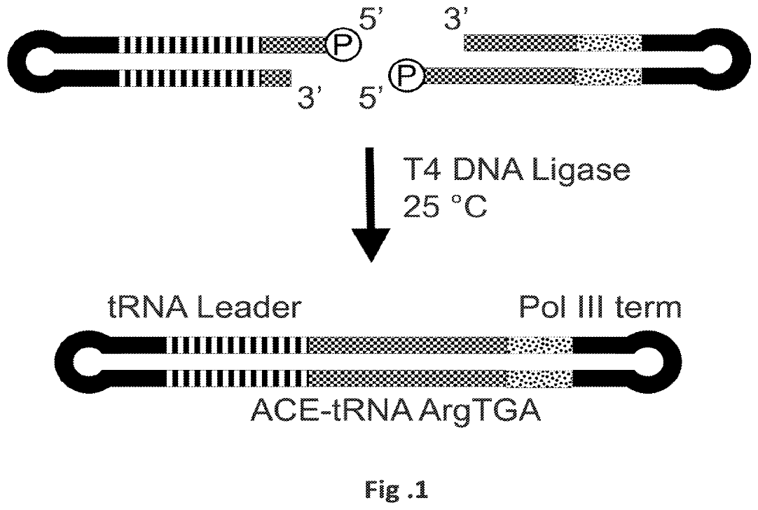

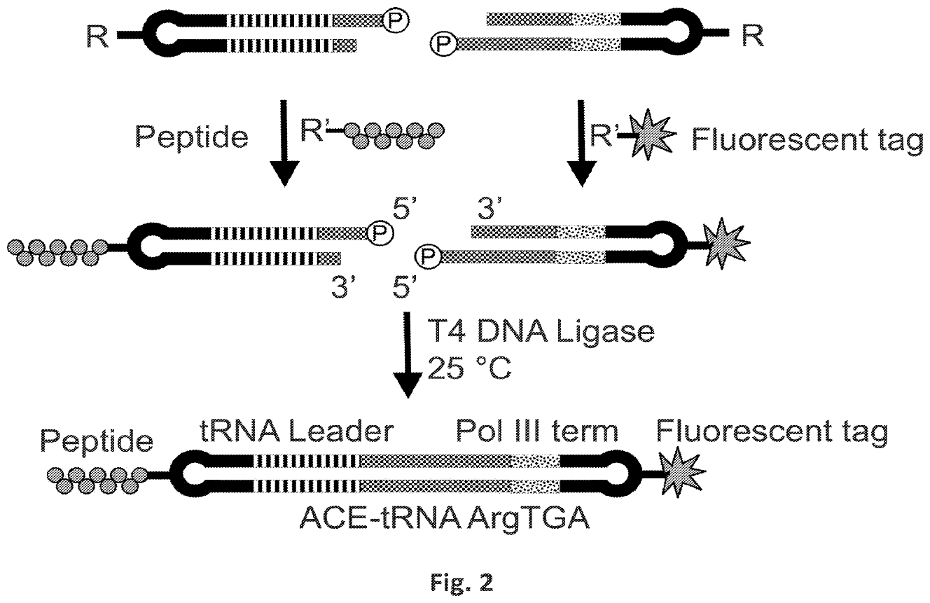

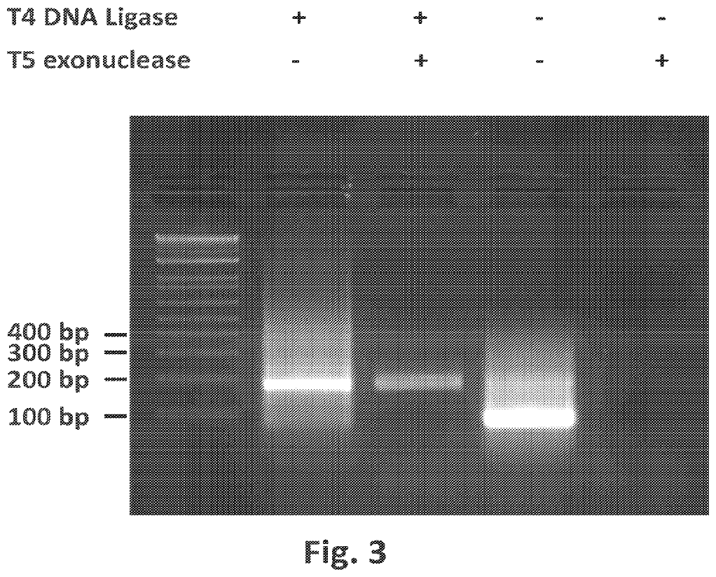

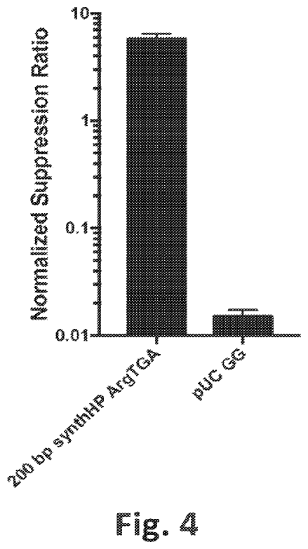

An example of the oligonucleotide set disclosed herein is illustrated in FIGS. 1 and 2.

As used herein, an “oligonucleotide” refers to a compound comprising a plurality of linked nucleosides. In some embodiments, an oligonucleotide may include one or more unmodified RNA and/or unmodified DNA and/or one or more modified nucleosides.

As used herein, a “hairpin,” “hairpin loop,” or “terminal hairpin” refers to a structure that forms when two regions of the same strand, usually complementary in nucleotide sequence when read in opposite directions, develop a pair of bases to form a double helix that ends in an unpaired loop.

As used herein, the terms such as “first,” “second,” and “third” are used to modify a noun, such use is simply intended to distinguish one item from another, and is not intended to require a sequential order unless specifically stated.

As used herein, an “antisense strand” refers to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

As used herein, the terms “complementary” or “complementarity” refer to “polynucleotides” and “oligonucleotides” (which are interchangeable terms that refer to a sequence of nucleotides) related by the base-pairing rules. For example, the sequence “C-A-G-T” is complementary to the sequence “G-T-C-A.” Complementarity can be “partial” or “total.” “Partial” complementarity is where one or more nucleic acid bases are not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, a “self-complementary sequence” refers to a first nucleic acid sequence on a first oligonucleotide, wherein a second oligonucleotide may include a second nucleic acid sequence in reverse order of the first nucleic acid. In this manner, the first and second nucleic acid sequences are complementary and may hybridize, thereby annealing the first and second oligonucleotides.

In some embodiments, the first hairpin may include a first segment of the nucleic acid sequence encoding a RNA molecule (e.g., ACE-tRNA), and the second hairpin may include a second segment of the nucleic acid sequence encoding a RNA molecule, such that when the first hairpin and second hairpin are jointed together, directly or indirectly, the first segment and the second segment of the nucleic acid sequence form a complete nucleic acid sequence that encodes a RNA molecule.

In some embodiments, when the first sense strand and the second sense strand are joined, the first hairpin oligonucleotide and the second hairpin oligonucleotide form a CEDT molecule. In some embodiments, the first sense strand and the second sense strand may be joined directly or indirectly, for example, by ligation.

As used herein, the term “ligate,” “ligating,” or “ligation” refers to any method or composition wherein two different double stranded nucleotides have been joined into a single oligonucleotide strand by a chemical reaction. Generally, a ligase enzyme (e.g., T4 DNA ligase, T3 DNA ligase) can be used to facilitate the joining process.

In some embodiments, the first sense strand and the second sense strand may be joined directly. For example, the first sense strand and the second sense strand may be joined directly through, e.g., ligatable ends, including blunt ends or sticky ends. In some embodiments, the first hairpin oligonucleotide and the second hairpin oligonucleotide may include a blunt end or a sticky end. To that end, the first antisense strand and first sense strand can have the same length or different lengths. Similarly, the second antisense strand and second sense strand can have the same length or different lengths.

As used herein, the term “blunt end” or “blunt-ended oligonucleotide” refers to an oligonucleotide that has a zero overhang. As used herein, the term “sticky end” refers to a double stranded polynucleotide molecule end that may include a sequence overhang. In some embodiments, the sticky end can be a nucleic acid molecule end with a 5′ or 3′ sequence overhang. In some embodiments, the sticky ends of the present disclosure are capable of hybridizing with compatible sticky ends of the same or other molecules. Thus, in some embodiments, a sticky end on the 3′ of a first DNA fragment may hybridize with a compatible sticky end on a second DNA fragment. In some embodiments, these hybridized sticky ends can be joined together by a ligase. In other embodiments, the sticky ends might require extension of the overhangs to complete the dsDNA molecule prior to ligation. In some embodiments, the first sense strand and the second sense strand may be joined indirectly through, for example, a spacer, such as a spacer nucleic acid sequence (e.g., double stranded DNA segment) that bridges the first sense strand and the second sense strand.

In some embodiments, the first hairpin oligonucleotide may include a nucleic acid sequence encoding a tRNA leader. For example, the tRNA leader may be encoded by a double-stranded DNA, and the sense strand and antisense strand of the double-stranded DNA may be located respectively on the first sense strand and the first antisense strand of the first hairpin oligonucleotide.

In some embodiments, the second hairpin oligonucleotide may include a nucleic acid sequence encoding a transcription termination signal. In some embodiments, the transcription termination signal is a RNA polymerase III termination signal.

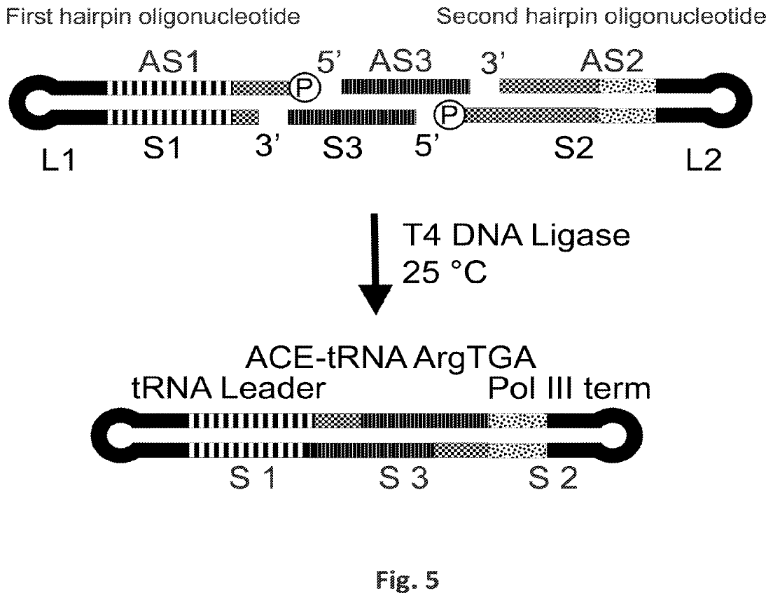

As shown in FIG. 5, the first hairpin oligonucleotide and the second hairpin oligonucleotide may also be joined together through one or more double stranded nucleic acid segments. Accordingly, in some embodiments, the oligonucleotide set may include a third sense strand and a third antisense strand having a sequence complementary to the third sense strand. In some embodiments, the first sense strand, the third sense strand, and the second sense strand are adapted to be joined together in the order to form a second nucleic acid sequence encoding the RNA molecule.

In another aspect, this disclosure provides an oligonucleotide set comprising: (i) a first hairpin oligonucleotide comprising from 5′ end to 3′ end: a first antisense strand, a first loop, and a first sense strand that is complementary to the first antisense strand; (ii) a second hairpin oligonucleotide comprising from 5′ end to 3′ end: a second sense strand, a second loop, and a second antisense strand that is complementary to the second sense strand; and (iii) a third sense strand and a third antisense strand having a sequence complementary to the third sense strand, wherein the first sense strand, the third sense strand, and the second sense strand are adapted to be joined together in the order to form a second nucleic acid sequence encoding the RNA molecule.

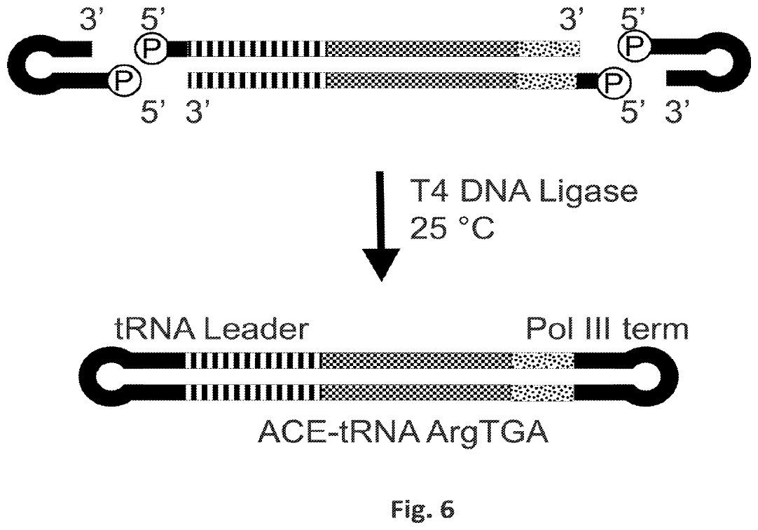

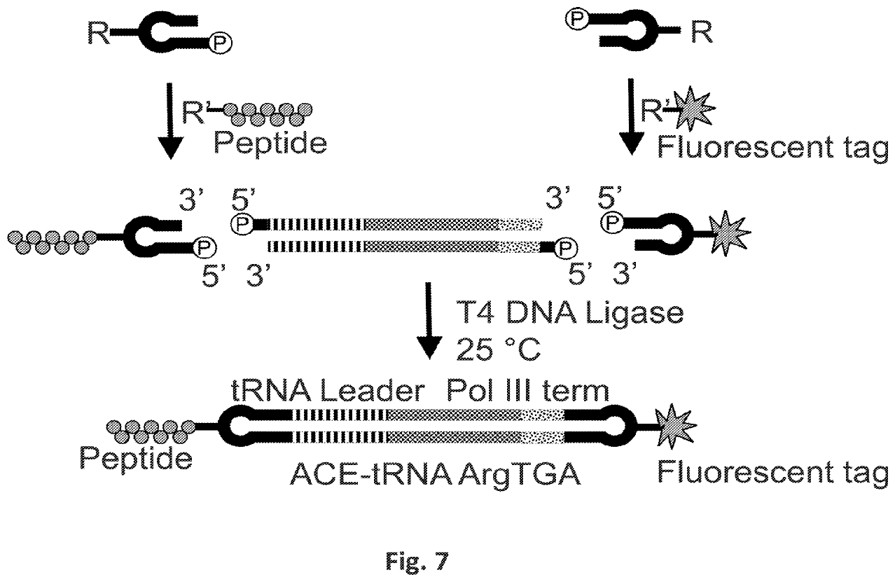

An example oligonucleotide set described herein is illustrated in FIGS. 6 and 7.

In some embodiments, when the first sense strand, the third sense strand, and the second sense strand are joined, the first hairpin oligonucleotide, the third sense and antisense strands, and the second hairpin oligonucleotide form a CEDT molecule.

### CEDT Molecules

As used herein, a “closed-end DNA thread,” “CEDT,” “CEDT molecule,” or “CEDT minivector” or “picovector” refers to a closed linear DNA molecule. Such a closed-end linear DNA molecule may be viewed as a single stranded circular molecule. A CEDT molecule may include covalently closed ends, also described as hairpin loops, where base-pairing between complementary DNA strands is not present. The hairpin loops join the ends of complementary DNA strands. Structures of this type typically form at the telomeric ends of chromosomes to protect against loss or damage of chromosomal DNA by sequestering the terminal nucleotides in a closed structure. In some examples of CEDT molecules described herein, hairpin loops flank complementary base-paired DNA strands, forming a “doggy-bone” shaped structure, e.g., as shown in FIGS. 1-2 and 5-7.

A CEDT molecule typically may include a linear double stranded section of DNA with covalently closed ends, i.e., hairpin ends. The hairpins join the ends of the linear double DNA strands, such that if the molecule was completely denatured, a single stranded circular DNA molecule would be produced. A CEDT, as described herein, can be essentially fully complementary in sequence, although some minor variations or “wobbles” may be tolerated by the structure. Thus, the closed linear DNA or CEDT may be at least 75%, 80%, 85%, 90%, or 95% complementary, or at least 96, 97, 98, 99 or 100% complementary in sequence. When denatured, it is effectively a circular molecule comprising both forward (sense or plus) and reverse (antisense or minus) strands adjacent to each other. This is in contrast to plasmid DNA or minicircle (MC) DNA where the complementary sequences (minus and plus) lie on separate circular strands.

The bases within the apex (end or turn) of the hairpin may not be able to form base pairs, due to the conformational stress put onto the DNA strand at this point. For example, at least two base pairs at the apex of the portion of the apex may not form base pairs, but the exact conformation is likely to be subject to fluctuations depending on the conditions in which the DNA is maintained and the exact sequences around the hairpin. Thus, despite their complementary nature, two or more bases may not be able to form pairs, given the structural distortion involved. Some “wobbles” of non-complementary bases within the length of a hairpin may not affect the structure. A wobble may be a break in the palindrome, but the sequences may remain complementary. In some embodiments, the sequence of a hairpin is entirely self-complementary. Complementarity describes how the bases of each polynucleotide in a sequence (5′ to 3′) are in a hydrogen-bonded pair with a complementary base, A to T (or U) and C to G on the anti-parallel (3′ to 5′) strand, which may be the same strand (internal complementary sequences) or on a different strand. This definition applies to any aspect or embodiment of the invention. In some embodiments, the sequences in the hairpin are 90% complementary, such as 91%, 92%, 93%, 94%, 95%, 96%, 98%, 99% or 100% complementary.

A CEDT may include any sequence within the double stranded sequence, either naturally derived or artificial. It may include at least one processing enzyme target sequence, such as one, two, three, four or more processing enzyme target sites. Such a target sequence is to allow for the DNA to be optionally processed further following synthesis. A processing enzyme is an enzyme that recognizes its target site and processes the DNA. The processing enzyme target sequence may be a target sequence for a restriction enzyme. A restriction enzyme, i.e., a restriction endonuclease, binds to a target sequence and cleaves at a specific point. The processing enzyme target sequence may be a target for a recombinase. A recombinase directionally catalyzes a DNA exchange reaction between short (30-40 nucleotides) target site sequences that are specific to each recombinase. Examples of recombinases include the Cre recombinase (with loxP as a target sequence) and FLP recombinase (with short flippase recognition target (FRT) sites). The processing enzyme target sequence may be a target for a site-specific integrase, such as the phiC31 integrase.

The processing enzyme target sequence may be a target sequence for a RNA polymerase, such that the CEDT becomes a template for RNA synthesis. In this instance, the processing enzyme targeting site is a promoter, such as a eukaryotic promoter. To that end, a CEDT may include an expression cassette comprising, consisting or consisting essentially of a eukaryotic promoter operably linked to a sequence enclosing a RNA (e.g., a tRNA) or protein of interest, and optionally a eukaryotic transcription termination sequence. A “promoter” is a nucleotide sequence which initiates and regulates transcription of a polynucleotide. “Operably linked” refers to an arrangement of elements wherein the components so described are configured so as to perform their usual function. Thus, a given promoter operably linked to a nucleic acid sequence is capable of effecting the expression of that sequence when the proper enzymes are present. The term “operably linked” is intended to encompass any spacing or orientation of the promoter element and the DNA sequence of interest, which allows for initiation of transcription of the DNA sequence of interest upon recognition of the promoter element by a transcription complex.

The CEDT may be of any suitable length. For example, the CEDT may have a size of up to 4 kb, such as 100 bp to 2 kb, 200 bp to 1 kb, or 200 bp to 800 bp. In some embodiments, CEDTs of 200 bp or longer can accommodate multiple ACE-tRNA cassettes/copies, allowing for higher ACE-tRNA expression from each CEDT unit. Having multiple copies of ACE-tRNAs from each CEDT enables one to include one or more sequences in each unit. For instance, a Leucine ACE-tRNA and a Tryptophan ACE-tRNA can be included in one CEDT molecule. Both of these ACE-tRNAs can be effective in cystic fibrosis for rescuing or suppressing the mutation W1282X-CFTR and significantly enhance suppression activity because they utilize different tRNA aminoacyl synthetases.

### ACE-tRNAs

In some embodiments, the tRNA may include an ACE-tRNA. An ACE-tRNA is an engineered tRNA molecule capable of reverting a PTC into the originally lost amino acid or a different amino acid. Such engineered tRNAs allow for “re-editing” of a disease-causing nonsense codon to a specific amino acid. The small size of these tRNA molecules makes them amenable to ready expression, as the tRNA and the promoter together can be only about 300 bp. To that end, an oligonucleotide can be synthesized to include the structural component of a tRNA gene that is functional in human cells. The sequence of this oligonucleotide can be designed based on a known sequence with substitutions made in the anticodon region of the tRNA, causing the specific tRNA to recognize nonsense or other specific mutations. Examples of ACE-tRNAs include those described in WO2019090154, WO 2019090169, WO2021252354A1, and Lueck, J. D. et al. Nature communications 10, 822 (2019), the contents of which are incorporated herein by reference.

Generally, an ACE-tRNA has a four-arm structure comprising a T-arm, a D-arm, an anticodon-arm, and an acceptor arm (see, e.g., FIG. 2 of WO2019090169). The T-arm is made up of a “T-stem” and a “TYE loop.” In some embodiments, the T-stem is modified to increase the stability of the tRNA. In some embodiments, the ACE-tRNA has a modified T-stem that increases the biological activity to suppress stop sites relative to the endogenous T-stem sequence.

ACE-tRNAs can be used for suppression of PTCs. This ACE-tRNA approach offers several significant benefits over other readthrough strategies, including (1) codon specificity; (2) ACE-tRNAs suppression of PTCs resulting in seamless rescue, thus negating spurious effects on protein stability, folding, trafficking, and function; and (3) in vitro delivery of these of ACE-tRNA resulting in significant functional rescue of affected protein, such as CFTR channels with p.G542X or p.W1282X CF mutations. The ACE-tRNAs have shown to be efficient at PTC suppression in several cDNA genes with varied PTC positions in multiple cell types. Because ACE-tRNAs exhibit high efficiency in PTC suppression with no known detrimental effects, they can be used as therapeutics.

ACE-tRNAs can be made according to the methods described in WO2019090154, WO2019090169, WO2021252354A1, and Lueck, J. D. et al., Nature communications 10, 822 (2019). Using the described methods, an extensive library of ACE-tRNAs for effective rescue of PTCs in cell culture can be generated. Other engineered human tRNA sequences to suppress disease-causing PTCs include those described in WO2019090154, WO2019090169, WO2021252354A1, and Lueck, J. D. et al., Nature communications 10, 822 (2019), the contents of which are incorporated herein by reference.

In some embodiments, ACE-tRNAs may be any one of Arg-tRNA-UGA, Gln-tRNA-UAA, GInt-RNA-UAG, Trp-tRNA-UGA, Trp-tRNA-UAG, Glu-tRNA-UAA, Glu-tRNA-UAG, Cys-tRNA-UGA, Tyr-tRNA-UAG, Tyr-tRNA-UAA, Leu-tRNA-UGA, Leu-tRNA-UAG, Leu-tRNA-UAA, Lys-tRNA-UAG, Lys-tRNA-UGA, Ser-tRNA-UGA, Ser-tRNA-UAG, and Ser-tRNA-UAA.

TABLE 1
Example nucleic acid sequences

| SEQ ID | | OTHER |
|---|---|---|
| NO | SEQUENCE | INFORMATION |
| 1 | /5Phos/TTGCGCCACAGAGCCGACGTGTACGTGTGT | ArgTGA synthHP |
| | CGGCGTCCCCTGAGGTTCAGCACAGAAAAACCG | oligo 1 |
| | GAGCGCTTAGAGTATAACTGCTCCATCCGCGGAT | |
| | GGAGCAGTTATACTCTAAGCGCTCCGGTTTTTCT | |
| | GTGCTGAACCTCAGGGGACGCCGACACACGTAC | |
| | ACGTCGGCTCTGTGGC | |
| 2 | /5Phos/GCAATGGATAGCGCATTGGACTTCAAATTC | ArgTGA synthHP |
| | AAAGGTTGTGGGTTCGAGTCCCACCAGAGTCGGT | oligo 2 |
| | CCTTTTTTTGATCCAGCAGGGTGGATGCCCGCGG | |
| | GCATCCACCCTGCTGGATCAAAAAAAGGACCGA | |
| | CTCTGGTGGGACTCGAACCCACAACCTTTGAATT | |
| | TGAAGTCCAATGCGCTATCCA | |
| 3 | /5Phos/TAGAGTCTACCTGCTCCATCCTCGGATGGA | STD5′HP |
| | GCAGGTAGACTCTAAGCGCTCCGGTTTTTCTGTG | |
| 4 | /5Phos/CTGAACCTCAGGGGACGCCGACACACGTA | Arg HP Fv2 |
| | CACGTCGGCTCTGTGGCGGCTCTGTGGCGCAATG | |
| | GATAGCGCATTGGACTTCAAATTCAAAGGTTGTG | |
| | GGTTCGAGTCCCACCAGAGTCG | |
| 5 | /5Phos/GGGAAAAAAAGGACCGACTCTGGTGGGAC | Arg HP Rv2 |
| | TCGAACCCACAACCTTTGAATTTGAAGTCCAATG | |
| | CGCTATCCATTGCGCCACAGAGCCGCCACAGAGC | |
| | CGACGTGTACGTGTGTCGGCGTCCCCTGAGGTTC | |
| | AGCACAGAAAAACCGGAGCGCT | |
| 6 | /5Phos/GTCCTTTTTTTCCCCATCAGTTTTTATAAAC | STD3′HP |
| | TTACACACTCGTGTGTAAGTTTATAAAAACTGAT | |
| | G | |
| 7 | /5Phos/CTCCAGGAGATAGAGTCTACCTGCTCCATC | STD5′HP v2 |
| | ctcgGATGGAGCAGGTAGACTCTA | |
| 8 | /5Phos/TCTCCTGGAGAGCGCTCCGGTTTTTCTGTG | ArgTGA HP Fv3 |
| | CTGAACCTCAGGGGACGCCGACACACGTACACGT | |
| | CGGCTCTGTGGCGGCTCTGTGGCGCAATGGATAG | |
| | CGCATTGGACTTCAAATTCAAAGGTTGTGGGTTC | |
| | GAGTCCCACCAGAGTCGGTCCTTTTTTT | |
| 9 | /5Phos/CCGTAGAGCGAAAAAAAGGACCGACTCTG | ArgTGA HP Rv3 |
| | GTGGGACTCGAACCCACAACCTTTGAATTTGAAG | |
| | TCCAATGCGCTATCCATTGCGCCACAGAGCCGCC | |
| | ACAGAGCCGACGTGTACGTGTGTCGGCGTCCCCT | |
| | GAGGTTCAGCACAGAAAAACCGGAGCGCT | |
| 10 | /5Phos/CGCTCTACGGGATCCAGCAGGGTGGATGC | STD3′HP v2 |
| | CCACActcgTGTGGGCATCCACCCTGCTGGATC | |
| 11 | /5Phos/CTCCAGGAGATAGAGTCTACCTGCTCCATC | DBCOS′HP |
| | c/DBCOTEG/cgGATGGAGCAGGTAGACTCTA | |
| 12 | /5Phos/CGCTCTACGGGATCCAGCAGGGTGGATGC | DBCO3′HP |
| | CCACAc/DBCOTEG/cgTGTGGGCATCCACCCTGCT | |
| | GGATC | |
| 13 | /5Phos/TCTCCTGGAGAGCGCTCCGGTTTTTCTGTG | LeuTGA HP Fv3 |
| | CTGAACCTCAGGGGACGCCGACACACGTACACGT | |
| | CACCAGAATGGCCGAGTGGTtAAGGCGTTGGACTt | |
| | caGATCCAATGGATTCATATCCGCGTGGGTTCGAA | |
| | CCCCACTTCTGGTAGTCCTTTTTTT | |
| 14 | /5Phos/CCGTAGAGCGAAAAAAAGGACTACCAGAA | LeuTGA HP Rv3 |
| | GTGGGGTTCGAACCCACGCGGATATGAATCCATT | |
| | GGATCtgaAGTCCAACGCCTTaACCACTCGGCCATT | |
| | CTGGTGACGTGTACGTGTGTCGGCGTCCCCTGAG | |
| | GTTCAGCACAGAAAAACCGGAGCGCT | |
| 15 | /5Phos/TCTCCTGGAGGCCAGGGGTGTGGCCATAC | OptArgTGA HP Fv3 |
| | AGGTTTATAGTGGTTAGTAGAGACAAGTAAGTGG | |
| | GTGCCCCTGTGGCGCAATGGATAGCGCATTGGAC | |
| | TtcaAATCCAAAGGTTGCGGGTTCGAGTCCCGCCA | |
| | GGGGCGGACCTTTTCCCCATCAGTTTTTATAAACT | |
| | TACACA | |
| 16 | /5Phos/CCGTAGAGCGTGTGTAAGTTTATAAAAACT | OptArgTGA HP Rv3 |
| | GATGGGGAAAAGGTCCGCCCCTGGCGGGACTCG | |
| | AACCCGCAACCTTTGGATTtgaAGTCCAATGCGCT | |
| | ATCCATTGCGCCACAGGGGCACCCACTTACTTGT | |
| | CTCTACTAACCACTATAAACCTGTATGGCCACAC | |
| | CCCTGGC | |
| 17 | /5Phos/TCTCCTGGAGGCCAGGGGTGTGGCCATAC | OptLeuTGA HP Fv3 |
| | AGGTTTATAGTGGTTAGTAGAGACAAGTAAGTGG | |
| | GTGCCAGGATGGCCGAGTGGTTAAGGCGCCGGA | |
| | CTtcaGATCCGGTGGACTCATGTCCGCCCGGGTTC | |
| | GAACCCCGGTCCTGGCAGACCTTTTCCCCATCAG | |
| | TTTTTATAAACTTACACA | |
| 18 | /5Phos/CCGTAGAGCGTGTGTAAGTTTATAAAAACT | OptLeuTGA HP Rv3 |
| | GATGGGGAAAAGGTCTGCCAGGACCGGGGTTCG | |
| | AACCCGGGCGGACATGAGTCCACCGGATCtgaAGT | |
| | CCGGCGCCTTAACCACTCGGCCATCCTGGCACCC | |
| | ACTTACTTGTCTCTACTAACCACTATAAACCTGTA | |
| | TGGCCACACCCCTGGC | |
| 19 | AGCGCTCCGGTTTTTCTGTGCTGAACCTCAGGGGAC | ArgTGA cassette |
| | GCCGACACACGTACACGTCGGCTCTGTGGCGCAAT | sequence (just the |
| | GGATAGCGCATTGGACTTCAAATTCAAAGGTTGT | active sequence |
| | GGGTTCGAGTCCCACCAGAGTCGGTCCTTTTTTT | assembled from the |
| | | oligos). oArgTGA |
| | | cassette components - |
| | | tRNA-Tyr-GTA-5-1 5′ |
| | | UCE - original ACE- |
| | | tRNA ArgTGA - |
| | | original 3′ trailer |
| 20 | AGCGCTCCGGTTTTTCTGTGCTGAACCTCAGGGGAC | LeuTGA cassette |
| | GCCGACACACGTACACGTCACCAGAATGGCCGAG | sequence (just the |
| | TGGTAAGGCGTTGGACTtcaGATCCAATGGATTCA | active sequence |
| | TATCCGCGTGGGTTCGAACCCCACTTCTGGTA | assembled from the |
| | GTCCTTTTTTT | oligos). oLeuTGA |
| | | cassette components - |
| | | tRNA-Tyr-GTA-5-1 5′ |
| | | UCE - original ACE- |
| | | tRNA LeuTGA - |
| | | original 3′ trailer |
| 21 | GCCAGGGGTGTGGCCATACAGGTTTATAGTGGTTAG | OptArgTGA cassette |
| | TAGAGACAAGTAAGTGGGTGCCCCTGTGGCGCAAT | sequence (just the |
| | GGATAGCGCATTGGACTtcaAATCCAAAGGTTGCG | active sequence |
| | GGTTCGAGTCCCGCCAGGGGCGGACCTTTTCCC | assembled from the |
| | CATCAGTTTTTATAAACTTACACA | oligos). |
| | | OptArgTGA cassette |
| | | components - tRNA- |
| | | Cys-GCA-12-1 5′ UCE |
| | | - optimized ACE- |
| | | tRNA ArgTGA - |
| | | optimized 3′ trailer |
| 22 | GCCAGGGGTGTGGCCATACAGGTTTATAGTGGTTAG | OptLeuTGA cassette |
| | TAGAGACAAGTAAGTGGGTGCCAGGATGGCCGAGT | sequence (just the |
| | GGTTAAGGCGCCGGACTtcaGATCCGGTGGACTCA | active sequence |
| | TGTCCGCCCGGGTTCGAACCCCGGTCCTGGCAGA | assembled from the |
| | CCTTTTCCCCATCAGTTTTTATAAACTTACACA | oligos). OptLeuTGA |
| | | cassette components - |
| | | tRNA-Cys-GCA-12-1 |
| | | 5′ UCE - optimized |
| | | ACE-tRNA LeuTGA - |
| | | optimized 3′ trailer |
| 23 | GCCAGGGGTGTGGCCATACAGGTTTATAGTGGTT | RNA leader sequence |
| | AGTAGAGACAAGTAAGTGGGT | (also referred to as |
| | | “upstream control |
| | | element” or “UCE”) |
| 24 | GACCTTTTCCCCATCAGTTTTTATAAACTTACACA | Termination signal |

Note:
SEQ ID Nos: 3, 4, 5, 6 can be annealed together and ligated to form the ArgTGA CEDT v1
SEQ ID Nos: 7, 8, 9, 10 can be annealed together and ligated to form the ArgTGA CEDT v2 (similar to v1 but the annealing overhangs and the 3′ hairpin have been changed)
SEQ ID Nos: 11 or 12 can be labeled with a fluorophore, peptide, or biotin, etc. using azide- alkyne “click” reaction, annealed with any of SEQ ID Nos: 8/9, 13/14, 15/16, or 17/18 and ligated to form labeled CEDTs v2 of either ArgTGA, LeuTGA, OptArgTGA, or OptLeuTGA
SEQ ID Nos: 7 and 10 can be annealed with any of SEQ ID Nos: 13/14, 15/16, or 17/18 and ligated to form unlabeled CEDTs v2 of either LeuTGA, OptArgTGA, or OptLeuTGA

In some embodiments, the third sense strand may include a polynucleotide sequence of SEQ ID NO: 4, 8, 13, 15, and 17, or may include a polynucleotide sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%) sequence identity with a polynucleotide sequence of SEQ ID NO: 4, 8, 13, 15, and 17.

In some embodiments, the third antisense strand may include a polynucleotide sequence of SEQ ID NO: 5, 9, 14, 16, and 18, or may include a polynucleotide sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%) sequence identity with a polynucleotide sequence of SEQ ID NO: 5, 9, 14, 16, and 18.

In some embodiments, the nucleic acid sequence may include a polynucleotide sequence of SEQ ID NO: 4, 8, 13, 15, and 17, or may include a polynucleotide sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%) sequence identity with a polynucleotide sequence of SEQ ID NO: 4, 8, 13, 15, and 17.

In some embodiments, the second nucleic acid sequence may include a polynucleotide sequence of SEQ ID NO: 4, 8, 13, 15, and 17, or may include a polynucleotide sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%) sequence identity with a polynucleotide sequence of SEQ ID NO: 4, 8, 13, 15, and 17.

In some embodiments, the first hairpin oligonucleotide may include a polynucleotide sequence of SEQ ID NO: 1, 3, 7, and 11, or may include a polynucleotide sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%) sequence identity with a polynucleotide sequence of SEQ ID NO: 1, 3, 7, and 11.

In some embodiments, the second hairpin oligonucleotide may include a polynucleotide sequence of SEQ ID NO: 2, 6, 10, and 12, or may include a polynucleotide sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%) sequence identity with a polynucleotide sequence of SEQ ID NO: 2, 6, 10, and 12.

In some embodiments, CEDT molecules formed from the disclosed oligonucleotide sets may have a size of from 200 nucleotides (nts) to 1,000 nts (e.g., 200, 250, 300, 350, 400, 45, 500, 550, 600, 650, 700, 750, 800, 850, 900, 950, 1000 nts). In some embodiments, the ACE-tRNA-coding double stranded segments have a size of, e.g., less than 200 bp, less than 250 bp, less than 300 bp, less than 350 bp, less than 400 bp, less than 450 bp, less than 500 bp, less than 550 bp, less than 600 bp, less than 650 bp, less than 700 bp, less than 750 bp, less than 800 bp, less than 850 bp, less than 900 bp, or less than 950 bp.

In some embodiments, one or more of the first loop or/and the second loop or/and another part of the oligonucleotide is/are linked with an agent or different agents. In some embodiments, the agent or agents may include a labeling agent, a peptide, a bioactive agent, or a combination thereof.

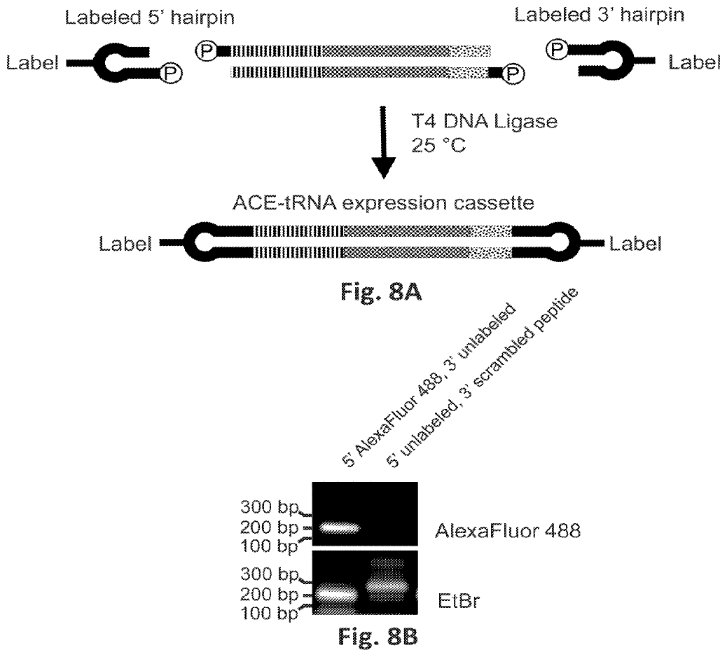

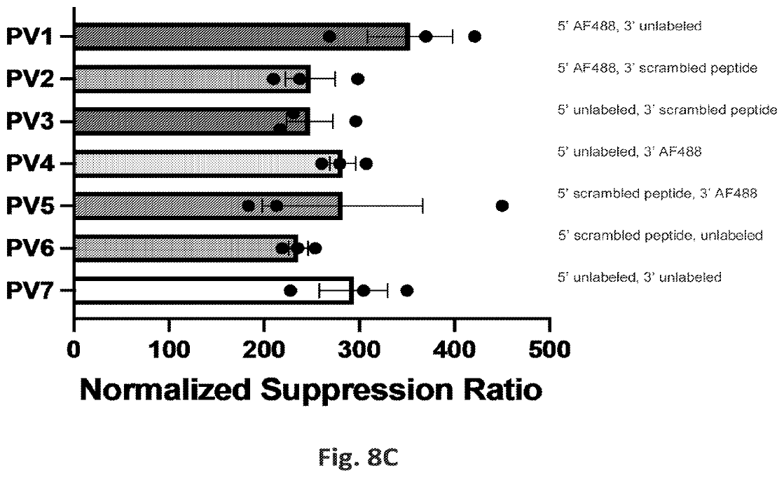

As used herein, the term “labeling agent,” “label,” or “detectable label” refers to any composition detectable by spectroscopic, photochemical, biochemical, immunochemical, electrical, optical, or chemical means. Such labels include biotin for staining with labeled streptavidin conjugate, magnetic beads (e.g., Dynabeads®), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. The use of such labels has been described in, e.g., U.S. Pat. Nos. 3,817,837; 3,850,752; 3,939,350, 3,996,345; 4,277,437; 4,275,149; and 4,366,241, the relevant contents of which are herein incorporated by reference. The labels may be detected by many methods. For example, radiolabels may be detected using photographic film or scintillation counters, and fluorescent markers may be detected using a photodetector to detect emitted light. Enzymatic labels are typically detected by providing the enzyme with a substrate and detecting the reaction product generated by the action of the enzyme on the substrate, and calorimetric labels are detected by simply visualizing the colored label. As shown in Example 3 and FIGS. 8A-8C, labeling or addition of modifications to the hairpin or CEDT or picovector do not negatively influence PTC suppression. Accordingly, such labeled hairpin or labeled CEDT/picovector can be used for various suitable applications such as identification of nuclear localization signal sequences and/or cell penetrating peptides that improve the picovector delivery and intracellular localization.

As used herein, the term “bioactive agent” refers to a substance that may be used in connection with an application that is therapeutic or diagnostic in nature, such as, for example, in methods for diagnosing the presence or absence of a disease in a patient or in methods for the treatment of disease in a patient.

In some embodiments, the labeling agent may include N-hydroxysuccinimide (NHS), thiol-maleimide, azide-dibenzocyclooctyne (DBCO), or a combination thereof.

In some embodiments, the nucleic acid molecules, such as the first hairpin oligonucleotide, the second hairpin oligonucleotide, or the CEDT molecules described herein, may include one or more chemically modified nucleotides, such as a 2′-O-methyl modified sugar moiety. For example, chemically modified nucleotides may include a modified internucleoside linkage. As used herein, a “modified oligonucleotide” refers to an oligonucleotide comprising at least one modified nucleoside and/or at least one modified internucleoside linkage. Examples of modified oligonucleotides include single-stranded and double-stranded compounds, such as antisense compounds, siRNAs, shRNAs, ssRNAs, and occupancy-based compounds.

As used herein, a “nucleoside” refers to a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety.

As used herein, a “chemical modification” refers to a chemical difference in a compound when compared to a naturally occurring counterpart. Chemical modifications of oligonucleotides may include nucleoside modifications (such as sugar moiety modifications and nucleobase modifications) and internucleoside linkage modifications. As used herein, an “internucleoside linkage” refers to a covalent linkage between adjacent nucleosides in an oligonucleotide. In reference to an oligonucleotide, a chemical modification does not include differences only in nucleobase sequence.

As used herein, a “sugar moiety” refers to a naturally occurring sugar moiety or a modified sugar moiety of a nucleoside. As used herein, a “modified sugar moiety” refers to a substituted sugar moiety or a sugar surrogate. As used herein, a “substituted sugar moiety” refers to a furanosyl that is not a naturally occurring sugar moiety. Substituted sugar moieties include, but are not limited to furanosyls comprising substituents at the 2′-position, the 3′-position, the 5′-position and/or the 4′-position. Certain substituted sugar moieties are bicyclic sugar moieties. As used herein, a “2′-substituted sugar moiety” refers to a furanosyl comprising a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring.

In some embodiments, chemical modifications may provide certain desirable properties, such as enhanced nuclease stability or increased binding affinity with a target nucleic acid relative to molecules having only nucleosides comprising naturally occurring sugar moieties. In some embodiments, modified sugar moieties are substituted sugar moieties. In some embodiments, modified sugar moieties are bicyclic or tricyclic sugar moieties. In some embodiments, modified sugar moieties are sugar surrogates. Such sugar surrogates may include one or more substitutions corresponding to those of substituted sugar moieties.

In some embodiments, modified sugar moieties are substituted sugar moieties comprising one or more substituents, including but not limited to substituents at the 2′ and/or 5′ positions. Examples of sugar substituents suitable for the 2′-position, include but are not limited to: 2′-F, 2′-OCH₃ (“OMe” or “O-methyl”), and 2′-O(CH₂)₂OCH₃ (“MOE”). In some embodiments, sugar substituents at the 2′ position are selected from allyl, amino, azido, thio, O-allyl, O—C₁-C₁₀ alkyl, O—C₁-C₁₀ substituted alkyl; O—C₁-C₁₀ alkoxy; O—C₁-C₁₀ substituted alkoxy, OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl. Examples of sugar substituents at the 5′-position include but are not limited to: 5′-methyl (R or S), S′-vinyl, and S′-methoxy. In some embodiments, substituted sugars may include more than one non-bridging sugar substituent, for example, 2′-F-5′-methyl sugar moieties (see, e.g., PCT International Application WO 2008/101157, for additional 5′,2′-bis substituted sugar moieties and nucleosides).

Nucleosides comprising 2′-substituted sugar moieties are herein referred to as 2′, substituted nucleosides. In some embodiments, a 2′-substituted nucleoside may include a 2′-substituent group selected from halo, allyl, amino, azido, O—C₁-C₁₀ alkoxy; O—C₁-C₁₀ substituted alkoxy, SH, CN, OCN, CF₃, OCF₃, O-alkyl, S-alkyl, N(Rₘ)-alkyl; O-alkenyl, S-alkenyl, or N(Rₘ)-alkenyl; O-alkynyl, S-alkynyl, N(Rₘ)-alkynyl; O-alkynyl-O-alkyl, alkynyl, alkaryl, aralkyl, O-alkaryl, O-aralkyl, O(CH₂)₂SCH₃, O—(CH₂)₂—O—N(Rₘ) (Rₙ) or O—CH₂—C(═O) N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl, and alkynyl.

In some embodiments, a 2′-substituted nucleoside may include a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂) zSCH₃, O—(CH₂)₂—O—N(Rₘ)(Rₙ), CH═CH₂, O(CH₂)₃O(CH₂)₂N(CH₃)₂, and N-substituted acetamide (O—CH₂C(═O)—N(Rₘ)(Rₙ) where each Rₘ and Rₙ is, independently, H, an amino protecting group or substituted or unsubstituted C₁-C₁₀ alkyl. In some embodiments, a 2′-substituted nucleoside may include a sugar moiety comprising a 2′-substituent group selected from F, OCF₃, O—CH₃, OCH₂CH₂OCH₃, O(CH₂)₂SCH₃, O—(CH₂)₂, —O—N(CH₃)₂, —O(CH)₂O(CH₂)₂N(CH₃)₂, and O—C₂—C(═O)—N(H)CH₃. In some embodiments, a 2′-substituted nucleoside may include a sugar moiety comprising a 2′-substituent group selected from F. O—CH₃, and OCH₂CH₂OCH₃.

In some embodiments, modified sugar moieties may include a bridging sugar substituent that forms a second ring resulting in a bicyclic sugar moiety. In some embodiments, the bicyclic sugar moiety may include a bridge between the 4′ and the 2′ furanose ring atoms Examples of such 4′ to 2′ sugar substituents, include, but are not limited to: —[C(Rₐ)(R_{b})]ₙ—, —[C(Rₐ)(R_{b})]ₙ—O—, —C(RₐR_{b})—N(R)—O— or, —C(RₐR_{b})—O—N(R)₄′—CH₂-2′,4′-(CH₂)₂-2′,4′-(CH₂)₃-2′,4′-(CH₂)—O-2′(LNA); 4′-(CH₂)—S-2; 4′-(CH₂)₂—O-2′ (ENA); 4′-CH(CH₃-O-2′ (cEt) and 4′-CH(CH₂OCH₃)—O-2′, and analogs thereof (see, e.g., U.S. Pat. No. 7,399,845, issued on Jul. 15, 2008); 4′-C(CH₃)(CH₃)—O-2′ and analogs thereof, (see, e.g., WO2009/006478, published Jan. 8, 2009); 4′-CH₂—N(OCH₃)-2′ and analogs thereof (see, e.g., WO2008/150729, published Dec. 11, 2008); 4′-CH₂—O—N(CH₃)-2′ (see, e.g., US2004/0171570, published Sep. 2, 2004); 4′-CH₂—O—N(R)-2′, and 4′-CH₂—N(R)-0-2′-, wherein each R is, independently, H, a protecting group, or C₁-C₁₂ alkyl; 4′-CH₂—N(R)—O-2′, wherein R is H, C₁-C₁₂ alkyl, or a protecting group (see, U.S. Pat. No. 7,427,672, issued on Sep. 23, 2008); 4′-CH₂—C(H)(CH₃)-2′ (see, e.g., Chattopadhyaya, el al., J. Org. Chem., 2009, 74, 118-134); and 4′-CH₂—C(═CH₂)-2′ and analogs thereof (see, published PCT International Application WO 2008/154401, published on Dec. 8, 2008).

### Methods for Making CEDTs

In yet another aspect, this disclosure further provides a method for making a CEDT molecule. In some embodiments, the method may include: providing an oligonucleotide set described herein; and ligating components of the oligonucleotide set, thereby obtaining the closed-end DNA thread molecule. In some embodiments, ligation of components of the oligonucleotide set may be assisted by an enzyme, such as a ligase. A ligase can be a eukaryotic ligase. The ligase can be a prokaryotic ligase. The ligase can be a single-stranded ligase. The ligase can be a double-stranded ligase. The ligase can be a DNA ligase. The DNA ligase can be T4 DNA ligase, Taq DNA ligase, T7 DNA ligase, T3 DNA ligase, 9° N™ DNA Ligase, and E. coli DNA ligase.

In some embodiments, the nucleic acid sequence may include a polynucleotide sequence of SEQ ID NO: 4, 8, 13, 15, and 17, or may include a polynucleotide sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%) sequence identity with a polynucleotide sequence of SEQ ID NO: 4, 8, 13, 15, and 17.

In some embodiments, the second nucleic acid sequence may include a polynucleotide sequence of SEQ ID NO: 4, 8, 13, 15, and 17, or may include a polynucleotide sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%) sequence identity with a polynucleotide sequence of SEQ ID NO: 4, 8, 13, 15, and 17.

In some embodiments, the first hairpin oligonucleotide may include a polynucleotide sequence of SEQ ID NO: 1, 3, 7, and 11, or may include a polynucleotide sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%) sequence identity with a polynucleotide sequence of SEQ ID NO: 1, 3, 7, and 11.

In some embodiments, the second hairpin oligonucleotide may include a polynucleotide sequence of SEQ ID NO: 2, 6, 10, and 12, or may include a polynucleotide sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%) sequence identity with a polynucleotide sequence of SEQ ID NO: 2, 6, 10, and 12.

In some embodiments, the oligonucleotide set may be synthesized chemically. In some embodiments, the oligonucleotide set may be synthesized with chemically modified nucleotides. In some embodiments, the first hairpin oligonucleotide, the second hairpin oligonucleotide, or the CEDT molecules may include one or more chemically modified nucleotides, such as a 2′-O-methyl modified sugar moiety. For example, chemically modified nucleotides may include a modified internucleoside linkage.

In some embodiments, the closed-end DNA thread molecule is further linked to a labeling agent, a peptide, a bioactive agent, or a combination thereof.

### Compositions and Kits

The nucleic acid molecules, such as oligonucleotide sets, hairpin oligonucleotides, or CEDT molecules generated from the disclosed oligonucleotide sets, can be provided in a composition (e.g., a pharmaceutical composition) or in a kit. In some embodiments, the composition may include an oligonucleotide set described herein. In some embodiments, the composition may include the first hairpin oligonucleotide and/or the second hairpin oligonucleotide, as described herein. In some embodiments, the composition may include a CEDT molecule prepared from an oligonucleotide set described herein.

Formulation of nucleic acids (e.g., DNA) as a conventional pharmaceutical preparation may be done using standard pharmaceutical formulation chemistries and methodologies available to those skilled in the art. Any pharmaceutically acceptable carrier or excipient may be used. Auxiliary substances, such as wetting or emulsifying agents, pH buffering substances, and the like, may be present in the excipient or vehicle. These excipients, vehicles, and auxiliary substances are generally pharmaceutical agents which may be administered without undue toxicity and which, in the case of vaccine compositions, will not induce an immune response in the individual receiving the composition. A suitable carrier may be a liposome. Pharmaceutically acceptable excipients include, but are not limited to, liquids such as water, saline, polyethylene glycol, hyaluronic acid, glycerol, and ethanol. Pharmaceutically acceptable salts can also be included therein, for example, mineral acid salts such as hydrochlorides, hydrobromides, phosphates, sulfates, and the like, and the salts of organic acids such as acetates, propionates, malonates, benzoates, and the like. The preparation may include a pharmaceutically acceptable excipient that serves as a stabilizer, particularly for peptide, protein, or other like molecules if they are to be included in the composition. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols (PEGs), or a combination thereof. A thorough discussion of pharmaceutically acceptable excipients, vehicles, and auxiliary substances is available in Remington's Pharmaceutical Sciences (Mack Pub. Co., N.J. 1991), incorporated herein by reference.

Compositions containing active ingredients, such as oligonucleotides or CEDT molecules, can be prepared by procedures known in the art using well-known and readily available ingredients. With respect to CEDT molecules, the compositions can also be formulated as solutions appropriate for parenteral administration, for instance, by intramuscular, subcutaneous, or intravenous routes. The compositions can be in the form of an aqueous or anhydrous solution or dispersion or in the form of an emulsion or suspension. Alternatively, compositions may be in powder form, obtained by aseptic isolation of sterile solid or by lyophilization from solution, for constitution with a suitable vehicle, e.g., sterile, pyrogen-free water, before use.

In some embodiments, the compositions disclosed herein may be formulated as lipid nanoparticles (LNP), such as those described in WO2020263883, WO2013123523, WO2012170930, WO2011127255, WO2008103276, and US20130171646, each of which is herein incorporated by reference in its entirety. Accordingly, the present disclosure provides nanoparticle compositions comprising a lipid composition comprising at least one nucleic acid, such as a CEDT molecule, and a delivery agent. In such a nanoparticle composition, the lipid composition disclosed herein can encapsulate the nucleic acid.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles, liposomes, and lipoplexes. In some embodiments, nanoparticle compositions are vesicles including one or more lipid bilayers. In some embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, a lipid nanoparticle may include an ionizable lipid, a structural lipid, a phospholipid, and nucleic acid of interest. In some embodiments, the lipid nanoparticle may include an ionizable lipid, a PEG-modified lipid, a sterol, and a structural lipid. In some embodiments, the lipid nanoparticle has a molar ratio of about 20-60% ionizable lipid: about 5-25% structural lipid: about 25-55% sterol; and about 0.5-15% PEG-modified lipid. In some embodiments, the lipid nanoparticle has a polydispersity value of less than 0.4. In some embodiments, the lipid nanoparticle has a net neutral charge at a neutral pH. In some embodiments, the lipid nanoparticle has a mean diameter of 50-150 nm. In some embodiments, the lipid nanoparticle has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, polyketides, and prenol lipids. In some instances, the amphiphilic properties of some lipids lead them to form liposomes, vesicles, or membranes in aqueous media.

In some embodiments, the nucleic acids can be formulated in lipid nanoparticles having a diameter from about 10 to about 100 nm. In some embodiments, the nanoparticles have a diameter from about 10 to 500 nm. In some embodiments, the nanoparticle has a diameter greater than 100 nm. In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter). As used herein, “size” or “mean size” in the context of nanoparticle compositions refers to the mean diameter of a nanoparticle.

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

In some embodiments, the nucleic acids described herein can be formulated for controlled release and/or targeted delivery. As used herein, “controlled release” refers to a pharmaceutical composition or compound release profile that conforms to a particular pattern of release to effect a therapeutic outcome. In one embodiment, the nucleic acids can be encapsulated into a delivery agent described herein and/or known in the art for controlled release and/or targeted delivery. As used herein, the term “encapsulate” means to enclose, surround or encase. As it relates to the formulation of the nucleic acids of this disclosure, encapsulation can be substantial, complete or partial. The term “substantially encapsulated” means that at least greater than 50, 60, 70, 80, 85, 90, 95, 96, 97, 98, 99, or greater than 99% of the pharmaceutical composition or nucleic acids of this disclosure can be enclosed, surrounded or encased within the delivery agent. “Partially encapsulation” means that less than 10, 10, 20, 30, 40, 50, or less of the pharmaceutical composition or nucleic acids of this disclosure can be enclosed, surrounded or encased within the delivery agent.

In some embodiments, the composition can be formulated for sustained release. As used herein, “sustained release” refers to a pharmaceutical composition or compound that conforms to a release rate over a specific period of time. The period of time can include, but is not limited to, hours, days, weeks, months, and years. As a non-limiting example, the sustained release nanoparticle composition described herein can be formulated as disclosed in WO2010075072, US20100216804, US20110217377, US20120201859, and US20130150295, each of which is herein incorporated by reference in their entirety. In some embodiments, the nanoparticle composition can be formulated to be target specific, such as those described in WO2008121949, WO2010005726, WO2010005725, WO2011084521 WO2011084518, US20100069426, US20120004293, and US20100104655, each of which is herein incorporated by reference in its entirety.

The nucleic acid molecules (e.g., oligonucleotide sets, hairpin oligonucleotides, CEDT molecules) or the composition thereof, as described herein, can be provided in a kit. In some embodiments, the kit includes a container that contains at least one nucleic acid molecule, or a composition thereof, and, optionally, informational material. The informational material can be descriptive, instructional, marketing, or other material that relates to the methods described herein and/or the use of the agents for therapeutic benefit. For example, kits may include instructions for manufacturing, the therapeutic regimen to be used, and periods of administration. In some embodiments, the kit may also include an additional therapeutic agent. The kit may include one or more containers, each with a different reagent. For example, the kit may include a first container that contains the composition and a second container for the additional agent, such as a therapeutic agent.

The containers may include a unit dosage of the pharmaceutical composition. In addition to the composition, the kit can include other ingredients, such as a solvent or buffer, an adjuvant, a stabilizer, a preservative, or a combination thereof. The kit may optionally include a device suitable for administration of the composition, e.g., a syringe or other suitable delivery device. The device may be provided pre-loaded with one or both of the agents or can be empty, but suitable for loading.

### Methods of Treatment

In yet another aspect, this disclosure additionally provides a method of treating a disease or disorder associated with PTCs in a mammal (such as a human). In some embodiments, the method may include administering to the mammal a CEDT molecule prepared from the disclosed oligonucleotide sets.

The disclosed method is advantageous because it provides improved stop codon suppression specificity. The therapeutic ACE-tRNAs of this disclosure can target a specific stop-codon, such as TGA, thus reducing off-target effects at stop-codons unrelated to disease. It is also advantageous because it provides amino acid specificity. The expressed tRNA is engineered to specifically replace the amino acid that was lost via insertion of the disease-associated stop codon, thus negating any spurious effects on protein stability, folding, and trafficking. Further, the method can be “personalized” to correct every possible disease PTC. For instance, there are nine individual tryptophan tRNAs in the human genome that are recognized by the Trp synthetase, all of which suppress the mRNA UGG codon. Thus, each of these nine Trp tRNAs provides an opportunity for codon re-editing tolerance (e.g., for UGG, UGA). Additionally, given their proximity to stop codons in the genetic code, the mutation of arginine codons to PTC nonsense codons are common in PTC-associated diseases. There are over thirty Arg tRNAs that can be used, and an ACE-tRNA that encodes Arginine is a viable therapeutic for all Arg->PTC mutations regardless of genes. Indeed, 35% of Leber congenital amaurosis (LCA) is caused by nonsense mutations, and the majority of the nonsense mutations are Arginine to stop codons. A further advantage of the disclosed method is that it provides facile expression and cell-specific delivery, because the entire system (tRNA+promoter sequence) is compact.

Diseases or disorders caused by or associated with PTCs include, but are not limited to, variants of Duchenne muscular dystrophies and Becker muscular dystrophies due to a PTC in dystrophin, retinoblastoma due to a PTC in RBI, neurofibromatosis due to a PTC in NF1 or NF2, ataxia-telangiectasia due to a PTC in ATM, Tay-Sachs disease due to a PTC in HEXA, cystic fibrosis due to a PTC in CFTR, Wilm's tumor due to a PTC in WT1, hemophilia A due to a PTC in factor VIII, hemophilia B due to a PTC in factor IX, p53-associated cancers due to a PTC in p53, Menkes disease, Ullrich's disease, b-thalassemia due to a PTC in betaglobin, type 2A and type 3 von Willebrand disease due to a PTC in Willebrand factor, Robinow syndrome, brachydactyly type B (shortening of digits and metacarpal s), inherited susceptibility to mycobacterial infection due to a PTC in IFNGR1, inherited retinal disease due to a PTC in CRX, inherited bleeding tendency due to a PTC in Coagulation factor X, inherited blindness due to a PTC in Rhodopsin, congenital neurosensory deafness and colonic agangliosis due to a PTC in SOXIO and inherited neural devel op-mental defect including neurosensory deafness, colonic agangliosis, peripheral neuropathy and central dysmyelinating leukodystrophy due to a PTC in SOX 10, Liddle's syndrome, xeroderma pigmentosum, Fanconi's anemia, anemia, hypothyroidism, p53-associated cancers (e.g., p53 squamal cell carcinoma, p53 hepatocellular carcinoma, p53 ovarian carcinoma), esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes, and rickets, etc.

In some embodiments, the method may include treating a disease or disorder, such as cystic fibrosis, by reversing the effects of mutations present that are associated with nonsense mutations through a CEDT molecule of this disclosure. Other diseases or disorders may include Hurler Syndrome, Dravet Syndrome, Spinal Muscular Dystrophy, Usher Syndrome, Aniridia, Choroideremia, Ocular Coloboma, Retinitis pigmentosa, dystrophic epidermolysis bullosa, Pseudoxanthoma elasticum, Alagille Snydrome, Waardenburg-Shah, infantile neuronal ceroid lipofuscinosis, Cystinosis, X-linked nephrogenic diabetes insipidus, and Polycystic kidney disease.

Additional diseases or disorders associated with PTCs that can be treated by the disclosed methods may include eye diseases. Examples of eye diseases may include those associated with one or more mutations in genes, including: Cone dystrophies (Stargardt's disease (STGD1), cone-rod dystrophy, retinitis pigmentosa (RP), and increased susceptibility to age-related macular degeneration): KCNV2 Glut 43 X; KCNV2 Glu306X; KCNV2 Gln76X; KCNV2 Glul48X; CACNA2D4, Tyr802X; CACNA2D4, Arg628X; RP2, Arg120X; Rho, Ser334X; Rpe65, Arg44X; PDE6A, Lys455X; Congenital stationary night blindness 2 (CSNB2): CACNAIF, Arg958X; CACNAIF, Arg830X; Congenital stationary night blindness 1 (CSNB1): TRPMl, Glnl1X; TRPMl, Lys294X; TRPMl, Arg977X; TRPMl, Ser882X; NYX, W350X; Best Disease or BVMD, BEST1, Tyr29X; BEST1, Arg200X; BEST1, Ser517X; Leber congenital amaurosis (LCA): KCNJ13, Trp53X; KCNJ13, Arg166X; CEP290, Arg151X; CEP290, Glyl890X; CEP290, Lys1575X; CEP290, Argl271X; CEP290, Arg1782X; CRB1, Cys1332X; GUCY2D, Ser448X; GUCY2D, Arg41091X; LCA5, Gln279X; RDH12, Tyr194X; RDH12, Glu275X; SPATA7, Arg108X; TULP1, Gln301X; Usher syndrome 1: USHIC, Arg31X; PCDH15, Arg3X; PCDH15, Arg245X; PCDH15, Arg643X; PCDH15, Arg929X; IQCB1, Arg461X; IQCB1, Arg489X; PDE6A, Gln69X; ALMSI, Ser999X; ALMSI, Arg3804X; Aniridia: Pax6, Glyl94X; Ocular coloboma: Pax2, Arg139X; Lambl, Arg524X; and Choroideremia: REPI, Gln32X.

The compositions can be administered in one or more doses and by techniques well known to those skilled in the medical arts taking into consideration such factors as the age, sex, weight, and condition of the particular subject and the route of administration. The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

The agent or composition can be administered prophylactically or therapeutically. In therapeutic applications, the agents or compositions are administered to a subject in need thereof in an amount sufficient to elicit a therapeutic effect. An amount adequate to accomplish this is defined as a “therapeutically effective dose.” Amounts effective for this use will depend on, e.g., the particular composition of the composition regimen administered, the manner of administration, the stage and severity of the disease, the general state of health of the subject, and the judgment of the prescribing physician.

The agent or composition can be administered by methods well known in the art as described in Donnelly et al. (Ann. Rev. Immunol. 15:617-648 (1997)), U.S. Pat. Nos. 5,580,859, 5,703,055, and 5,679,647, the contents of which are incorporated herein by reference. The DNA of the composition can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector. The composition can be delivered via a variety of routes. Typical delivery routes include parenteral administration, e.g., intradermal, intramuscular, or subcutaneous delivery. Other routes include oral administration, intranasal, and intravaginal routes. The composition may be delivered to the interstitial spaces of tissues of an individual (U.S. Pat. Nos. 5,580,859 and 5,703,055, the contents of all of which are incorporated herein by reference in their entirety). The composition can also be administered to muscle, or can be administered via intradermal or subcutaneous injections, or transdermally, such as by iontophoresis. Epidermal administration of the composition can also be employed. Epidermal administration can involve mechanically or chemically irritating the outermost layer of epidermis to stimulate an immune response to the irritant (U.S. Pat. No. 5,679,647).

In some embodiments, the composition can be formulated for administration via the nasal passages. Formulations suitable for nasal administration, wherein the carrier is a solid, can include a coarse powder having a particle size, for example, in the range of about 10 to about 500 microns, which are administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the composition.

The composition can be a liquid preparation such as a suspension, syrup, or elixir. The composition can also be a preparation for parenteral, subcutaneous, intradermal, intramuscular, or intravenous administration (e.g., injectable administration), such as a sterile suspension or emulsion.

The composition can be incorporated into liposomes, microspheres, or other polymer matrices (U.S. Pat. No. 5,703,055; Gregoriadis, Liposome Technology, Vols. Ito III (2nd ed. 1993), the contents of which are incorporated herein by reference in their entirety). Liposomes can consist of phospholipids or other lipids and can be nontoxic, physiologically acceptable, and metabolizable carriers that are relatively simple to make and administer.

The composition may be administered by different routes, including orally, parenterally, sublingually, transdermally, rectally, transmucosally, topically, via inhalation, via buccal administration, intrapleurally, intravenously, intraarterially, intraperitoneally, subcutaneously, intramuscularly, intranasally, intrathecally, intraarticularly or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns,” or other physical methods such as electroporation (“EP”), “hydrodynamic method,” or ultrasound.

In some embodiments, the composition may be delivered to the mammal by several well-known technologies, including DNA injection with and without in vivo electroporation, liposome-mediated, nanoparticle facilitated, recombinant vectors such as recombinant adenovirus, recombinant adenovirus associated virus, and recombinant vaccinia. The ACE-tRNA or nucleic acid molecule encoding the ACE-tRNA may be delivered via DNA injection and along with in vivo electroporation.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

A nucleic acid or polynucleotide refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single stranded or double stranded. An “isolated nucleic acid” refers to a nucleic acid, and its structure is not identical to that of any naturally occurring nucleic acid or to that of any fragment of a naturally occurring genomic nucleic acid. The term, therefore, covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. The nucleic acid described above can be used to express the tRNA of this invention. For this purpose, one can operatively link the nucleic acid to suitable regulatory sequences to generate an expression vector.

As used herein, “translation” means the process in which a polypeptide (e.g., a protein) is translated from an mRNA. In some embodiments, an increase in translation means an increase in the number of polypeptide molecules (e.g., a protein) that are made per copy of mRNA that encodes said polypeptide.

As used herein, “non-complementary” in reference to nucleobases means a pair of nucleobases that do not form hydrogen bonds with one another.

As used herein, “mismatch” means a nucleobase of a first oligomeric compound that is not capable of pairing with a nucleobase at a corresponding position of a second oligomeric compound, when the first and second oligomeric compounds are aligned. Either or both of the first and second oligomeric compounds may be oligonucleotides.

As used herein, a “vector” refers to a nucleic acid molecule capable of transporting another nucleic acid to which it has been linked. The vector may or may not be capable of autonomous replication or integration into a host DNA. Examples of the vector include a plasmid, cosmid, or viral vector. The vector includes a nucleic acid in a form suitable for expression of a nucleic acid of interest in a host cell. Preferably the vector includes one or more regulatory sequences operatively linked to the nucleic acid sequence to be expressed.

As used herein, a “regulatory sequence” includes promoters, enhancers, and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein or RNA desired, and the like. The expression vector can be introduced into host cells to produce an RNA or a polypeptide of interest. A promoter is defined as a DNA sequence that directs RNA polymerase to bind to DNA and initiate RNA synthesis. A strong promoter is one which causes RNAs to be initiated at high frequency.

As used herein, a “promoter” is a nucleotide sequence that initiates and regulates transcription of a polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e. g., controls transcription or translation) segments of these regions.

As used herein, the term “operably linked” refers to an arrangement of elements wherein the components so described are configured so as to perform their usual function. Thus, a given promoter operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence. Thus, the term “operably linked” is intended to encompass any spacing or orientation of the promoter element and the DNA sequence of interest, which allows for initiation of transcription of the DNA sequence of interest upon recognition of the promoter element by a transcription complex.

As used herein, an “expression cassette” refers to a nucleic acid sequence capable of directing expression of a particular nucleotide sequence in an appropriate host cell, which may include a promoter operably linked to the nucleotide sequence of interest that may be operably linked to termination signals. It also may include sequences required for proper translation of the nucleotide sequence. The coding region usually codes for an RNA or protein of interest. The expression cassette, including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development. In some embodiments, the promoter is a PGK, CMV, RSV, HI or U6 promoter (Pol II and Pol III promoters). A “nucleic acid fragment” is a portion of a given nucleic acid molecule. The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%), or even at least 95%, 96%, 97%, 98%, or 99% sequence identity, compared to a reference sequence using one of the alignment programs described using standard parameters.

As used herein, a “minivector” refers to a mini-sized and circular DNA vector system, e.g., a double stranded circular DNA (e.g., a mini circle) or a closed linear DNA molecule (e.g., CEDT), lacking a bacterial origin of replication and an antibiotic selection gene, and having a size of about 100 bp up to about 5 kbp. It can be obtained, for example, by site-specific recombination of a parent plasmid to eliminate plasmid sequences outside of the recombination sites. It contains, for example, a nucleic acid molecule with merely the transgene expression cassette, including promoter and a nucleic acid sequence of interest, wherein the nucleic acid sequence may be, for example, an ACE-tRNA, e.g., for suppressing PTCs, and importantly, no bacterial-originated sequences.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, a “subject” or “subject in need thereof” refers to a human and a non-human animal. Examples of a non-human animal include all vertebrates, e.g., mammals, such as non-human mammals, non-human primates (particularly higher primates), dog, rodent (e.g., mouse or rat), guinea pig, cat, and rabbit, and non-mammals, such as birds, amphibians, reptiles, etc. In some embodiments, the subject is a human. In another embodiment, the subject is an experimental animal or animal suitable as a disease model.

A disease or disorder associated with a PTC or nonsense mutation, PTC-associated disease, or PTC-associated disease refers to any conditions caused or characterized by one or more nonsense mutations that change an amino acid codon to PTC through a single-nucleotide substitution, resulting in a defective truncated protein.

As used herein, “treating” or “treatment” refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder. The terms “prevent,” “preventing,” “prevention,” “prophylactic treatment,” and the like refer to reducing the probability of developing a disorder or condition in a subject, who does not have, but is at risk of or is susceptible to developing a disorder or condition. “Ameliorating” generally refers to the reduction in the number or severity of signs or symptoms of a disease or disorder.

As used herein, the terms “prevent,” “preventing,” and “prevention” refer generally to a decrease in the occurrence of disease or disorder in a subject. The prevention may be complete, e.g., the total absence of the disease or disorder in the subject. The prevention may also be partial, such that the occurrence of the disease or disorder in the subject is less than that which would have occurred without embodiments of the present invention. “Preventing” a disease generally refers to inhibiting the full As used herein, the of a disease.

As used herein, the term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active compound. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate.

As used herein, the term “agent” refers to a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

As used herein, the terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, the term “pharmaceutically acceptable” refers to a material, such as a carrier or diluent, which does not abrogate the biological activity or properties of the composition and is relatively non-toxic, i.e., the material may be administered to an individual without causing undesirable biological effects or interacting in a deleterious manner with any of the components of the composition in which it is contained.

As used herein, the term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24:307-331, which is herein incorporated by reference. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Conservative amino acid substitution groups include: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions, and other modifications, including conservative amino acid substitutions. For instance, GCG software contains programs such as GAP and BESTFIT, which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters. A program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and (1997) Nucleic Acids Res. 25:3389-3402, each of which is herein incorporated by reference.

Doses are often expressed in relation to body weight. Thus, a dose which is expressed as [g, mg, or other unit]/kg (or g, mg, etc.) usually refers to [g, mg, or other unit] “per kg (or g, mg, etc.) bodyweight,” even if the term “bodyweight” is not explicitly mentioned.

As used herein, the term “in vitro” refers to events that occur in an artificial environment, e.g., in a test tube or reaction vessel, in cell culture, etc., rather than within a multicellular organism.

As used herein, the term “in vivo” refers to events that occur within a multicellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “In some embodiments,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition that is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. When used in this document, the term “exemplary” is intended to mean “by way of example” and is not intended to indicate that a particular exemplary item is preferred or required.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

This example describes materials and methods used in subsequent examples.

ArgTGA synthHP Production Conditions:
a. SynthHP oligo annealing conditions

SynthHP oligo 1 and oligo 2 were delivered as desalted oligos (Integrated DNA Technologies (IDT)) and were dissolved in a T.E. buffer at 2 μg/uL and annealed in a 30 μL reaction containing 12 uL molecular biology grade H₂O, 15 uL dissolved oligo (2 μg/uL), and 3 uL 10× annealing buffer (1 M potassium acetate and 300 mM HEPES, pH 7.5). The annealing samples were heated to 95° C. in a thermocycler for 10 minutes and then cooled to 4° C. over 30 minutes.
b. SynthHP ligation reaction conditions

Each annealed oligo was added to the ligation reaction (10 uL annealed oligo, 100 ng/uL final concentration). The reaction also contained 10 uL 10× T4 DNA ligase buffer (New England Biolabs (NEB)), 5 uL T4 DNA ligase (NEB) or 5 uL H₂O for the no ligase control, and 65 uL H2O. The reaction was allowed to proceed overnight at room temperature.
c. T5 exonuclease reaction conditions:

5 uL T5 exonuclease (NEB) or 5 uL H₂O for the no T5 control was added to the completed ligation reactions, incubated at 37° C. for 1 hour, and then at 80° C. for 10 minutes. Following the T5 exonuclease reaction, the products were resolved on a 1% agarose gel (figure at left). A T5-resistant product appears in the plus ligase reaction indicating that the intact ArgTGA synthHP is formed in the ligation reaction, while in the no ligase control reaction following T5 exonuclease treatment, there was no product evident. The yield following T5 exonuclease treatment and anion exchange purification was approximately 4 μg synthHP for a 10 μg oligo input.

As the synthHP oligonucleotides can be chemically synthesized, any of a number of chemical modifications can be included. The synthHP allows for attachment of defined chemical moieties, such as bioorthogonal or other specific reactive groups, to the synthHP and the assembled ArgTGA synthHP CEDT. Reactive pairs, denoted herein as R—R′ pairs, can be used to ligate molecules of interest, and a retrosynthetic approach also allows attachment of two different chemical moieties with the same R—R′ pair. For instance, in this case, a cellular targeting peptide and fluorescent tag are covalently linked to the completed synthHP ArgTGA CEDT. The R—R′ pair can be any of amine-NHS (N-hydroxysuccinimide), thiol-maleimide, or azide-DBCO (Dibenzocyclooctyne), among others. This approach is amenable to adopting many commercially available labeling reagents.

### Example 2

This example shows ArgTGA synthHP functioned in vivo.

The ArgTGA synthHP or a pUC GG negative control plasmid were co-transfected into HEK293T cells along with a reporter plasmid (UbC-Fluc WT, CMV Nluc-TGA-PTC) using lipofectamine 2000 (Invitrogen) according to manufacturer protocols in a black 96-well cell culture plate. Following transfection, the cells were kept in a CO₂ incubator at 37° C. After a 24-hour incubation, the media was removed via aspiration, and 15 uL of PBS was added to the wells. The amount of Fluc and Nluc expression was determined using the Dual-Glo Assay kit (Promega) and measured using a Synergy2 multi-mode microplate reader (BioTek instruments). The data is presented as the Nluc signal from each well normalized to the Fluc signal from the same well. Data are presented as the average of 3 wells+standard error of the mean. The ArgTGA synthHP shows a rescue of 386-fold over the background.

### Example 3

This example shows that addition of modifications to a CEDT or picovector do not influence its PTC suppression.

More specifically, ACE-tRNA DNA picovectors were produced and labeled in the manner shown in FIG. 8A. Synthetic DNA hairpins corresponding to the 5′ and 3′ ends of the picovector, containing chemical functional groups for site specific labeling were pre-labeled and assembled in a T4 DNA ligase dependent reaction.

In one picovector product, the 5′ hairpin was labeled with Alexa Fluor 488 while the 3′ hairpin was not labeled. The labeled ACE-tRNA DNA picovector products were resolved by electrophoresis with the 5′-Alexa Fluor 488 (AF488) containing product exhibiting AF488 fluorescence. The results are shown in FIG. 8B. As shown in the figure, the band representing the picovector DNA was evident under conditions for imaging AF488.

In another picovector product, the 3′ hairpin was labeled with a scrambled peptide sequence: {AzidoLys}GGPSEVANKEPQQTAEGKEGKTRAKRDEDQ (SEQ ID NO: 25), while the 5′ hairpin was not labeled. The electrophoresis results are shown in FIG. 8B as well. As shown in the figure, the 3′-peptide labeled product exhibited an electrophoretic mobility shift where the band representing the picovector DNA was shifted upwards when imaged with EtBr.

To examine the efficiencies of the labeled ACE-tRNAArgTGA picovectors in nonsense suppression, the following labeled ACE-tRNAArgTGA picovectors were transfected into HEK293T cells, and the corresponding nonsense suppressions were examined in the manner described herein using a dual luciferase reporter system (firefly luciferase for transfection normalization, nanoluc-PTC to assay nonsense suppression.

| Picovectors | Modifications |
|---|---|
| PV1 | 5′ AF488, 3′ unlabeled |
| PV2 | 5′ AF488, 3′ scrambled peptide |
| PV3 | 5′ unlabeled, 3′ scrambled peptide |
| PV4 | 5′ unlabeled, 3′ AF488 |
| PV5 | 5′ scrambled peptide, 3′ AF488 |
| PV6 | 5′ scrambled peptide, 3′ unlabeled |
| PV7 | 5′ unlabeled, 3′ unlabeled |

As shown in FIG. 8C, each of the labeled picovectors exhibited similar nonsense suppression efficiency when assayed. These results suggest that addition of modifications do not negatively influence PTC suppression.

The foregoing examples and description of the preferred embodiments should be taken as illustrating, rather than as limiting the present disclosure as defined by the claims. As will be readily appreciated, numerous variations and combinations of the features set forth above can be utilized without departing from the present disclosure as set forth in the claims. Such variations are not regarded as a departure from the scope of the disclosure, and all such variations are intended to be included within the scope of the following claims. All references cited herein are incorporated by reference in their entireties.

1. An oligonucleotide set comprising:
a first hairpin oligonucleotide comprising from 5′ end to 3′ end:
a first antisense strand,
a first loop, and
a first sense strand that is complementary to the first antisense strand; and
a second hairpin oligonucleotide comprising from 5′ end to 3′ end:
a second sense strand,
a second loop, and
a second antisense strand that is complementary to the second sense strand,
wherein the first sense strand and the second sense strand are adapted to be joined together to form a nucleic acid sequence encoding a RNA molecule.

2. An oligonucleotide set comprising:
a first hairpin oligonucleotide comprising from 5′ end to 3′ end:
a first antisense strand,
a first loop, and
a first sense strand that is complementary to the first antisense strand;
a second hairpin oligonucleotide comprising from 5′ end to 3′ end:
a second sense strand,
a second loop, and
a second antisense strand that is complementary to the second sense strand; and
a third sense strand and a third antisense strand having a sequence complementary to the third sense strand,
wherein the first sense strand, the third sense strand, and the second sense strand are adapted to be joined together in the order to form a second nucleic acid sequence encoding a RNA molecule.

3. The oligonucleotide set of claim 1, wherein when the first sense strand and the second sense strand are joined, the first hairpin oligonucleotide and the second hairpin oligonucleotide form a closed-end DNA thread (CEDT) molecule.

4. The oligonucleotide set of claim 1, wherein the first hairpin oligonucleotide comprises a nucleic acid sequence encoding a tRNA leader.

5. The oligonucleotide set of claim 1, wherein the second hairpin oligonucleotide comprises a nucleic acid sequence encoding a RNA polymerase III termination signal.

6. The oligonucleotide set of claim 1, further comprising a third sense strand and a third antisense strand having a sequence complementary to the third sense strand.

7. The oligonucleotide set of claim 6, wherein the first sense strand, the third sense strand, and the second sense strand are adapted to be joined together in the order to form a second nucleic acid sequence encoding the RNA molecule.

8. The oligonucleotide set of claim 2, wherein when the first sense strand, the third sense strand, and the second sense strand are joined, the first hairpin oligonucleotide, the third sense and antisense strands, and the second hairpin oligonucleotide form a closed-end DNA thread (CEDT) molecule.

9. The oligonucleotide set of claim 2, wherein the third sense strand comprises a nucleic sequence encoding a tRNA leader or a RNA polymerase III termination signal.

10. (canceled)

11. The oligonucleotide set of claim 1, wherein the RNA molecule comprises IRNA.

12. The oligonucleotide set of claim 1, wherein the tRNA comprises an anti-code edited-tRNA (ACE-IRNA).

13. The oligonucleotide set of claim 11, wherein the tRNA is selected from the group consisting of Arg-tRNA-UGA, Gln-tRNA-UAA, GInt-RNA-UAG, Trp-tRNA-UGA, Trp-tRNA-UAG, Glu-tRNA-UAA, Glu-tRNA-UAG, Cys-tRNA-UGA, Tyr-tRNA-UAG, Tyr-tRNA-UAA, Leu-tRNA-UGA, Leu-tRNA-UAG, Leu-tRNA-UAA, Lys-IRNA-UAG, Lys-tRNA-UGA, Ser-tRNA-UGA, Ser-tRNA-UAG, and Ser-tRNA-UAA.

14. (canceled)

15. (canceled)

16. (canceled)

17. The oligonucleotide set of claim 1, wherein the nucleic acid sequence or the second nucleic acid sequence comprises a polynucleotide sequence of SEQ ID NO: 4, 8, 13, 15, and 17; or comprises a polynucleotide sequence having at least 85% sequence identity with a polynucleotide sequence of SEQ ID NO: 4, 8, 13, 15, and 17.

18. The oligonucleotide set of claim 1, wherein:
the first hairpin oligonucleotide comprises a polynucleotide sequence of SEQ ID NO: 1, 3, 7, and 11, or comprises a polynucleotide sequence having at least 85% sequence identity with a polynucleotide sequence of SEQ ID NO: 1, 3, 7, and 11; and/or
the second hairpin oligonucleotide comprises a polynucleotide sequence of SEQ ID NO: 2, 6, 10, and 12, or comprises a polynucleotide sequence having at least 85% sequence identity with a polynucleotide sequence of SEQ ID NO: 2, 6, 10, and 12.

19. (canceled)

20. The oligonucleotide set of any one of claim 1, wherein the first loop or the second loop or another part of the oligonucleotide is linked with an agent or
wherein the first hairpin oligonucleotide or the second hairpin oligonucleotide comprises one or more chemically modified nucleotides.

21. (canceled)

22. (canceled)

23. (canceled)

24. (canceled)

25. (canceled)

26. A composition or a kit comprising the oligonucleotide set of claim 1.

27. (canceled)

28. (canceled)

29. A method for making a closed-end DNA thread (CEDT) molecule, comprising:
providing an oligonucleotide set according to claim 1; and
ligating components of the oligonucleotide set, thereby obtaining the CEDT.

30. (canceled)

31. (canceled)

32. (canceled)

33. A closed-end DNA thread (CEDT) molecule made according to the method of claim 29.

34. A method of treating a disease associated with a premature termination codon (PTC) in a subject in need thereof, comprising administering to the subject the closed-end DNA thread (CEDT) molecule of claim 33 or a pharmaceutical composition thereof.

35. The method of claim 34, wherein the disease is selected from the group consisting of cystic fibrosis, Duchenne and Becker muscular dystrophies, retinoblastoma, neurofibromatosis, ataxia-telangiectasia, Tay-Sachs disease, Wilm's tumor, hemophilia A, hemophilia B, Menkes disease, Ullrich's disease, b-Thalassemia, type 2A and type 3 von Willebrand disease, Robinow syndrome, brachydactyly type B (shortening of digits and metacarpals), inherited susceptibility to mycobacterial infection, inherited retinal disease, inherited bleeding tendency, inherited blindness, congenital neurosensory deafness and colonic agangliosis and inherited neural develop-mental defect including neurosensory deafness, colonic agangliosis, peripheral neuropathy and central dysmyelinating leukodystrophy, Liddle's syndrome, xeroderma pigmentosum, Fanconi's anemia, anemia, hypothyroidism, p53-associated cancers, esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes, rickets, Hurler Syndrome, Dravet Syndrome, Spinal Muscular Dystrophy, Usher Syndrome, Aniridia, Choroideremia, Ocular Coloboma, Retinitis pigmentosa, dystrophic epidermolysis bullosa, Pseudoxanthoma elasticum, Alagille Syndrome, Waardenburg-Shah, infantile neuronal ceroid lipofuscinosis, Cystinosis, X-linked nephrogenic diabetes insipidus, McArdle's disease and Polycystic kidney disease.
